(19) United States
(12) Patent Application Publication
(10) Pub. No.: US 2009/0269764 A1
(43) Pub. Date: Oct. 29, 2009

(54) COMPOSITIONS AND METHODS FOR DETECTION OF PROPIONIBACTERIUM ACNES NUCLEIC ACID

(75) Inventors: James J. HOGAN, Coronado, CA (US); Kristin W. Livezey, Encinitas, CA (US); Shannon K. Kaplan, San Diego, CA (US); Jennifer J. Bungo, San Diego, CA (US)

Correspondence Address: GEN PROBE INCORPORATED, 10210 GENETIC CENTER DRIVE, Mail Stop #1 / Patent Dept., SAN DIEGO, CA, 92121, US

(73) Assignee: Gen-Probe Incorporated, San Diego, CA (US)

(21) Appl. No.: 12/389,350

(22) Filed: Feb. 19, 2009

Related U.S. Application Data
(60) Provisional application No. 61/029,849, filed on Feb. 19, 2008.

(51) Int. Cl. C12Q 1/68

(52) U.S. Cl. 435/6

(57) ABSTRACT

Methods for amplifying and detecting Propionibacterium acnes nucleic acid by targeting specific sequences in 16S rRNA, 23S rRNA, or DNA encoding 16S rRNA or 23S rRNA are disclosed. Nucleic acid oligonucleotide sequence compositions specific for P. acnes nucleic acid sequences in 16S or 23S rRNA or DNA encoding 16S or 23S rRNA sequences are disclosed, which are useful for amplification oligonucleotides, capture probes in sample preparation, and probes for detection of P. acnes nucleic acid sequences.

### RELATED APPLICATION

This application claims the benefit under 35 U.S.C. 119(e) of provisional application No. 61/029,849, filed Feb. 19, 2008, which is incorporated by reference.

### FIELD OF THE INVENTION

This invention relates to detection of bacteria in a sample by using molecular biological methods and more specifically relates to detection of the presence of Propionibacterium acnes in a sample by amplifying nucleic acids from P. acnes and detecting the amplified nucleic acid sequences.

### BACKGROUND

Propionibacterium acnes is a gram positive, non-spore-forming, rod-shaped bacterium. P. acnes is part of the normal flora of the skin, oral cavity, large intestine and conjunctiva. P. acnes accounts for approximately half of the total skin microbiota with an estimated density of 102 to 106 organisms per square centimeter (J. Clin. Microbiol. 2005; 43:326). Although P. acnes is mainly associated with acne vulgaris, it has also been associated with a number of inflammatory conditions, post operative disorders and opportunistic infections in immunosuppressed hosts (Lett. Applied Microbiol. 2006; 42:185, Anaerobe 2003; 9:5).

P. acnes is a fastidious organism that is difficult to culture, requiring special equipment and expertise for anaerobic culture. Routine cultures often miss P. acnes, especially when a less fastidious aerobic bacterium is present. Also, the presence of P. acnes in clinical samples is often dismissed as contamination when other medically important bacteria are present (J. Med. Microbiol. 2004; 53:1247). P. acnes is slow growing (e.g., forming colonies less than one millimeter after four days) and, therefore, samples suspected of containing P. acnes should be cultured for up to two weeks.

Due to the abundance of P. acnes on human skin, it can become a contaminant in any process that requires the presence of people. In many industries, it is important to keep bacterial contaminants below a certain level. Materials, such as water, used to make products for the food, drugs, diagnostics and cosmetics industries are tested for contaminants throughout the manufacturing process. Water used in drug, device or diagnostic manufacturing for the USA must meet United States Pharmacopia (USP) specifications limiting the amount of chemical and microbial contaminants. The USP purity standards for drugs, devices and diagnostics are enforceable by the U.S. Food and Drug Administration. For example, the USP 23 standard limits microbial contamination, measured as colony forming units (CFU), to less than or equal to 100 CFU per milliliter of purified water.

There is a need for a rapid, sensitive and accurate method to detect P. acnes, particularly in manufacturing samples so that a contaminating source is accurately detected and then eliminated. There is also a need for methods that allow rapid and accurate diagnosis of P. acnes infections in humans to allow prompt and effective treatment of infected individuals and minimize further infections.

### SUMMARY

A composition comprising at least two oligomers specific for a P. acnes 16S rRNA sequence or DNA encoding 16S rRNA contained within SEQ ID NO: 199 selected from the group consisting of SEQ ID Nos. 16 to 26, 35 to 54, 135 to 160, 184, and 210, the completely complementary sequences of SEQ ID Nos. 16 to 26, 35 to 54, 135 to 160, 184, and 210, and the RNA equivalent of SEQ ID Nos. 16 to 26, 35 to 54, 135 to 160, 184, and 210, wherein when a first oligomer of the at least two oligomers is selected from the group consisting of SEQ ID Nos. 16 to 26, 135 to 160, 184 and 210, then a second oligomer is selected from the group consisting of SEQ ID Nos. 35 to 54. One embodiment of the composition further comprises at least one third oligomer selected from the group consisting of SEQ ID Nos. 1 to 8, 75 to 98, 127 to 130, 181, 182, 195 to 198, and 211 to 226, the completely complementary sequences of SEQ ID Nos. 1 to 8, 75 to 98, 127 to 130, 181, 182, 195 to 198, and 211 to 226, and the RNA equivalents of SEQ ID Nos. 1 to 8, 75 to 98, 127 to 130, 181, 182, 195 to 198, and 211 to 226. Another embodiment of the composition of includes a promoter provider oligomer selected from the group consisting of SEQ ID Nos. 35 to 54 and 135 to 160. One embodiment of the composition that includes at least one third oligomer includes an oligomer selected from the group consisting of SEQ ID Nos. 1 to 8, the completely complementary sequences of SEQ ID Nos. 1 to 8, and the RNA equivalent of SEQ ID Nos. 1 to 8, for which the third oligomer selected from this group includes a 3′ blocked end. In another embodiment, the at least one third oligomer is selected from the group consisting of SEQ ID Nos. 75 to 98, 181, and 182, the completely complementary sequences of SEQ ID Nos. 75 to 98, 181, and 182, and the RNA equivalent of SEQ ID Nos, 75 to 98, 181, and 182, for which the third oligomer selected from this group includes a label attached to the third oligomer.

A composition comprising at least two oligomers specific for a P. acnes 23S rRNA sequence or DNA encoding 23S rRNA contained within SEQ ID NO:202 selected from the group consisting of SEQ ID Nos. 27 to 34, 55 to 72, 161 to 180, and 185 to 187, the completely complementary sequences of SEQ ID Nos. 27 to 34, 55 to 72, 161 to 180, and 185 to 187, and the RNA equivalent of SEQ ID Nos. 27 to 34, 55 to 72, 161 to 180, and 185 to 187, wherein when a first oligomer of the at least two oligomers is selected from the group consisting of SEQ ID Nos. 27 to 34, 161 to 180, and 185, then a second oligomer is selected from the group consisting of SEQ ID Nos. 55 to 72, 186, and 187. In one embodiment, the composition further includes at least one third oligomer selected from the group consisting of SEQ ID Nos. 9 to 15, 99 to 126, 131 to 134, 183, and 188, the completely complementary sequences of SEQ ID Nos. 9 to 15, 99 to 126, 131 to 134, 183, and 188, and the RNA equivalent of SEQ ID Nos. 9 to 15, 99 to 126, 131 to 134, 183, and 188. In another embodiment of the composition, the at least two oligomers include a promoter provider oligomer selected from the group consisting of SEQ ID Nos. 55 to 72, 186, and 187, the completely complementary sequences of SEQ ID Nos. 55 to 72, 186, and 187, and the RNA equivalent of SEQ ID Nos. 55 to 72, 186, and 187. In an embodiment of the composition that includes at least one third oligomer, it is selected from the group consisting of SEQ ID Nos. 9 to 15 and 188, the completely complementary sequences of SEQ ID Nos. 9 to 15 and 188, and the RNA equivalent of SEQ ID Nos. 9 to 15 and 188, and the third oligomer selected from this group includes a 3′ blocked end. In another embodiment, the at least one third oligomer is selected from the group consisting of SEQ ID Nos. 99 to 126 and 183, the completely complementary sequences of SEQ ID Nos. 99 to 126 and 183, and the RNA equivalent of SEQ ID Nos. 99 to 126 and 183, for which the third oligomer selected from this group includes a label attached to the third oligomer.

A method for specifically detecting a P. acnes in a sample by detecting a target nucleic acid sequence that is a P. acnes rRNA or DNA encoding a P. acnes rRNA comprising the steps of: providing a sample that contains a P. acnes target region in a 16S rRNA or DNA encoding 16S rRNA contained in SEQ ID NO:199, and/or a target region in a 23S rRNA or gene encoding 23S RNA contained in SEQ ID NO:202; amplifying a sequence in the P. acnes target region to make an amplified product by using at least two amplification oligomers in an in vitro amplification reaction that includes enzymatic elongation of at least one amplification oligomer, wherein when the target sequence is in the 16S rRNA or DNA encoding the 16S rRNA contained in SEQ ID NO: 199, the at least two amplification oligomers are selected from the group consisting of SEQ ID Nos. 16 to 26, 35 to 54, 135 to 160, 184, and 210, the completely complementary sequences of SEQ ID Nos. 16 to 26, 35 to 54, 135 to 160, 184, and 210, and the RNA equivalent of SEQ ID Nos. 16 to 26, 35 to 54, 135 to 160, 184, and 210, and wherein when the target sequence is in the 23S rRNA or DNA encoding 23S rRNA contained in SEQ ID NO:202, the at least two amplification oligomers are selected from the group consisting of SEQ ID Nos. 27 to 34, 55 to 72, 161 to 180, and 185 to 187, the completely complementary sequences of SEQ ID Nos. 27 to 34, 55 to 72, 161 to 180, and 185 to 187, and the RNA equivalent of SEQ ID Nos. 27 to 34, 55 to 72, 161 to 180, and 185 to 187; and detecting the amplified product. One embodiment of the method, in which the target sequence is in the 16S rRNA or DNA encoding the 16S rRNA, the amplification oligomers use a first amplification oligomer selected from the group consisting of SEQ ID Nos. 16 to 26, 135 to 160, 184 and 210, with a second amplification oligomer selected from the group consisting of SEQ ID Nos. 35 to 54. In an embodiment in which the amplifying step amplifies the target sequence in the 16S rRNA or DNA encoding the 16S rRNA, the method further includes a third oligomer selected from the group consisting of SEQ ID Nos. 1 to 8, the completely complementary sequences of SEQ ID Nos. 1 to 8, and the RNA equivalent of SEQ ID Nos. 1 to 8, for which the third oligomer selected from this group includes a 3′ blocked end. In one embodiment, the detecting step for the amplified product made from the target sequence in the 16S rRNA or DNA encoding the 16S rRNA uses a detection probe selected from the group consisting of SEQ ID Nos. 75 to 98, 181, and 182, the completely complementary sequences of SEQ ID Nos. 75 to 98, 181, and 182, and the RNA equivalent of SEQ ID Nos. 75 to 98, 181, and 182. In another embodiment, the method further includes a target capture step to purify the 16S rRNA or DNA encoding the 16S rRNA from the sample by hybridizing to the 16S rRNA or DNA encoding the 16S rRNA a target capture probe selected from the group consisting of SEQ ID Nos. 127 to 130. In one embodiment of the method for which the target sequence is in the 23S rRNA or DNA encoding the 23S rRNA, the two amplification oligomers use a first amplification oligomer selected from the group consisting of SEQ ID Nos. 27 to 34, 161 to 180, and 185, with a second amplification oligomer selected from the group consisting of SEQ ID Nos. 55 to 72, 186, and 187. In another embodiment, in which the amplifying step amplifies the target sequence in the 23S rRNA or DNA encoding the 23S rRNA, the amplifying step further includes a third oligomer selected from the group consisting of SEQ ID Nos. 9 to 15 and 188, the completely complementary sequences of SEQ ID Nos. 9 to 15 and 188, and the RNA equivalent of SEQ ID Nos. 9 to 15 and 188, for which the third oligomer selected from this group includes a 3′ blocked end. In one embodiment, the detecting step for the amplified product made from the target sequence in the 23S rRNA or DNA encoding the 23S rRNA uses a detection probe selected from the group consisting of SEQ ID Nos. 99 to 126 and 183, the completely complementary sequences of SEQ ID Nos. 99 to 126 and 183, and the RNA equivalent of SEQ ID Nos. 99 to 126 and 183. One embodiment of the method further includes a target capture step to purify the 23S rRNA or DNA encoding the 23S rRNA from the sample by hybridizing to the 23S rRNA or DNA encoding the 23S rRNA a target capture probe selected from the group consisting of SEQ ID Nos. 131 to 134. In an embodiment of the method, the amplified product is detected by using a detection probe that includes a label.

### DETAILED DESCRIPTION

Disclosed are methods for detecting the presence or absence of P. acnes in a sample, including food, water, industrial, environmental or biological specimens. These methods provide for the sensitive and specific detection of P. acnes nucleic acids. The methods include performing nucleic acid amplification of the 16S or 23S rRNA sequences specific for P. acnes and detecting the amplified product. In some embodiments, detection occurs by specifically hybridizing the amplified product with a nucleic acid probe that provides a signal to indicate the presence of P. acnes in the sample. Absence of P. acnes in the sample is detected in an embodiment of the method includes an internal control that is a non-P. acnes nucleic acid in the reaction mixture that treated for simultaneous amplification of a P. acnes target nucleic acid, but in which no amplified P. acnes product is made while an amplified product is made for the internal control in the reaction mixture. That is, amplification of the internal control in the reaction mixture indicates that the method steps were properly performed and the reaction conditions were functional for making amplified nucleic acid but no P. acnes amplified product was made because a P. acnes target nucleic acid was not present in the tested sample.

Amplification includes contacting the sample with a one or more amplification oligonucleotides specific for a target sequence in a ribosomal RNA (rRNA) or DNA encoding an rRNA, more specifically 16S or 23S rRNA or DNA encoding 16S or 23S rRNA, to produce an amplified product if the P. acnes target nucleic acid is present in the sample. Amplification synthesizes additional copies of the target sequence or its complement by using at least one nucleic acid polymerase to extend the sequence from an amplification oligomer (e.g., a primer) by using a template strand that is part of the target nucleic acid or its complementary strand. One embodiment detects the amplified product by using a hybridizing step that includes contacting the amplified product with at least one probe specific for a sequence amplified by the amplification oligonucleotides, e.g., a sequence contained in the target sequence flanked by a pair of selected amplification oligomers.

Detecting the amplified products may be performed after the amplification reaction is completed, or may be performed simultaneous with amplifying the target region, i.e., in real time. In one embodiment, the detection step allows homogeneous detection, i.e., detection of the hybridized probe to the amplified product without removal of unhybridized probe from the mixture (e.g., see U.S. Pat. Nos. 5,639,604 and 5,283,174, Arnold Jr. et al.). In embodiments that detect the amplified product near or at the end of the amplification step, a linear probe may be used to provide a signal to indicate hybridization of the probe to the amplified product. In other embodiments that use real-time detection, the probe may be a hairpin probe, such as a molecular beacon, molecular torch, or hybridization switch probe, that is labeled with a reporter moiety that is detected when the probe binds to amplified product. For example, a hairpin probe may include a label, such as a fluorophore (“F”), attached to one end of the probe and an interacting compound, such as quencher (“Q”), attached to the other end the hairpin structure to inhibit signal production from the label when the hairpin structure is in the “closed” conformation and not hybridized to the amplified product, whereas a signal is detectable when the probe is in the “open” conformation because it is hybridized to a complementary sequence in the amplified product. Various forms of such probes are known (e.g., see U.S. Pat. Nos. 5,118,801 and 5,312,728, Lizardi et al., U.S. Pat. Nos. 5,925,517 and 6,150,097, Tyagi et al., U.S. Pat. Nos. 6,849,412, 6,835,542, 6,534,274, and 6,361,945, Becker et al., and US2006-0068417A1, Becker et al., and US2006-0194240A1, Arnold Jr.)

To aid in understanding aspects of this disclosure, some terms used herein are described in more detail. All other scientific and technical terms used herein have the same meaning as commonly understood by those skilled in the relevant art, such as may be provided in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), and references cited herein. Unless mentioned otherwise, the techniques employed or contemplated herein are standard methods well known to a person of ordinary skill in the art of molecular biology.

“Sample” includes any specimen that may contain P. acnes or components thereof, such as nucleic acids or fragments of nucleic acids. Samples may be obtained from environmental or manufacturing sources, e.g., water, soil, slurries, debris, biofilms from containers of aqueous fluids, airborne particles or aerosols, and the like, which may include processed samples, such as obtained from passing samples over or through a filtering device, or following centrifugation, or by adherence to a medium, matrix, or support. Samples include “biological samples” which include any tissue or material derived from a living or dead mammal or organism which may contain P. acnes or target nucleic acid derived therefrom, including, e.g., respiratory tissue or exudates such as bronchoscopy, bronchioalveolar lavage, lung biopsy, sputum, peripheral blood, plasma, serum, lymph node, gastrointestinal tissue, feces, urine, or other body fluids or materials. A sample may be treated to physically or mechanically disrupt tissue aggregates or cells, thus releasing intracellular components, including nucleic acids, into a solution which may contain other components, such as enzymes, buffers, salts, detergents and the like.

“Nucleic acid” refers to a multimeric compound comprising nucleosides or nucleoside analogs which have nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid “backbone” may be made up of a variety of linkages, including one or more of sugar-phosphodiester linkages, peptide-nucleic acid bonds (in “peptide nucleic acids” or PNAs, see PCT No. WO 95/32305), phosphorothioate linkages, methylphosphonate linkages, or combinations thereof. Sugar moieties of the nucleic acid may be either ribose or deoxyribose, or similar compounds having known substitutions, e.g., 2′ methoxy substitutions and 2′ halide substitutions (e.g., 2′-F). Nitrogenous bases may be conventional bases (A, G, C, T, U), analogs thereof (e.g., inosine, 5-methylisocytosine, isoguanine; The Biochemistry of the Nucleic Acids 5-36, Adams et al., ed., 11^{th} ed., 1992, Abraham et al., 2007, BioTechniques 43: 617-24), which include derivatives of purine or pyrimidine bases (e.g., N⁴-methyl deoxygaunosine, deaza- or aza-purines, deaza- or aza-pyrimidines, pyrimidine bases having substituent groups at the 5 or 6 position, purine bases having an altered or replacement substituent at the 2, 6 and/or 8 position, such as 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines, and pyrazolo-compounds, such as unsubstituted or 3-substituted pyrazolo[3,4-d]pyrimidine; U.S. Pat. Nos. 5,378,825, 6,949,367 and PCT No. WO 93/13121). Nucleic acids may include “abasic” residues in which the backbone does not include a nitrogenous base for one or more residues (U.S. Pat. No. 5,585,481, Arnold et al.). A nucleic acid may comprise only conventional sugars, bases, and linkages as found in RNA and DNA, or may include conventional components and substitutions (e.g., conventional bases linked by a 2′ methoxy backbone, or a nucleic acid including a mixture of conventional bases and one or more base analogs). Nucleic acids may include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA) (Vester et al., 2004, Biochemistry 43(42):13233-41). Synthetic methods for making nucleic acids in vitro are well known in the art although nucleic acids may be purified from natural sources using routine techniques.

The interchangeable terms “oligomer” and “oligonucleotide” refer to a nucleic acid having generally less than 1,000 nucleotide (nt) residues, including polymers in a range having a lower limit of about 5 nt residues and an upper limit of about 500 to 900 nt residues. In some embodiments, oligonucleotides are in a size range having a lower limit of about 12 to 15 nt and an upper limit of about 50 to 600 nt, and other embodiments are in a range having a lower limit of about 15 to 20 nt and an upper limit of about 22 to 100 nt. Oligonucleotides may be purified from naturally occurring sources, but or may be synthesized using any of a variety of well known enzymatic or chemical methods.

An “amplification oligomer” is an oligomer that hybridizes to a target nucleic acid, or its complement, and participates in a nucleic acid amplification reaction. An example of an amplification oligomer is a “primer” that hybridizes to a template nucleic acid and contains a 3′ OH end that is extended by a polymerase in an amplification process. Another example of an amplification oligomer is an oligomer that is not extended by a polymerase (e.g., because it has a 3′ blocked end) but participates in or facilitates amplification. Size ranges for amplification oligonucleotides include those that are about 10 to about 70 nt long (not including the promoter sequence or poly-A tails) and contain at least about 10 contiguous bases, or even at least 12 contiguous bases that are complementary to a region of the target nucleic acid sequence (or a complementary strand thereof). The contiguous bases are at least 80%, or at least 90%, or completely complementary to the target sequence to which the amplification oligomer binds. An amplification oligomer may optionally include modified nucleotides or analogs, or additional nucleotides that participate in an amplification reaction but are not complementary to or contained in the target nucleic acid, or template sequence. For example, the 5′ region of an amplification oligonucleotide may include a promoter sequence that is non-complementary to the target nucleic acid (which may be referred to as a “promoter-primer” or “promoter provider”). Those skilled in the art will understand that an amplification oligomer that functions as a primer may be modified to include a 5′ promoter sequence, and thus function as a promoter-primer. Similarly, a promoter-primer may be modified by removal of, or synthesis without, a promoter sequence and still function as a primer. In another example, an amplification oligomer that is 3′ blocked but capable of hybridizing to a target nucleic acid and providing an upstream promoter sequence that serves to initiate transcription is referred to as a “promoter provider” oligomer.

Oligomers that are not intended to be extended by a nucleic acid polymerase may include a blocker group that replaces the 3′OH to prevent enzyme-mediated extension of the oligomer in an amplification reaction. For example, blocked amplification oligomers and/or detection probes present during amplification may not have functional 3′OH and instead include one or more blocking groups located at or near the 3′ end. In some embodiments a blocking group near the 3′ end and may be within five residues of the 3′ end and is sufficiently large to limit binding of a polymerase to the oligomer. In other embodiments a blocking group is covalently attached to the 3′ terminus. Many different chemical groups may be used to block the 3′ end, e.g., alkyl groups, non-nucleotide linkers, alkane-diol dideoxynucleotide residues, and cordycepin.

“Amplification” refers to any known procedure for obtaining multiple copies of a target nucleic acid sequence or its complement or fragments thereof. Amplification of “fragments” refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), ligase chain reaction (LCR), strand-displacement amplification (SDA), and transcription-mediated or transcription-associated amplification. Replicase-mediated amplification uses self-replicating RNA molecules, and a replicase such as QB-replicase (e.g., U.S. Pat. No. 4,786,600, Kramer et al.). PCR amplification uses a DNA polymerase, pairs of primers, and thermal cycling to synthesize multiple copies of two complementary strands of dsDNA or from a cDNA (e.g., U.S. Pat. Nos. 4,683,195, 4,683,202, and 4,800,159, Mullis et al.). LCR amplification uses four or more different oligonucleotides to amplify a target and its complementary strand by using multiple cycles of hybridization, ligation, and denaturation (e.g., U.S. Pat. No. 5,427,930, Birkenmeyer et al., U.S. Pat. No. 5,516,663, Backman et al.). SDA uses a primer that contains a recognition site for a restriction endonuclease and an endonuclease that nicks one strand of a hemimodified DNA duplex that includes the target sequence, whereby amplification occurs in a series of primer extension and strand displacement steps (e.g., see U.S. Pat. Nos. 5,422,252, Walker et al., 5,547,861, Nadeau et al., and 5,648,211, Fraiser et al.).

“Transcription associated amplification” or “transcription mediated amplification” (TMA) refer to nucleic acid amplification that uses an RNA polymerase to produce multiple RNA transcripts from a nucleic acid template. These methods generally employ an RNA polymerase, a DNA polymerase, deoxyribonucleoside triphosphates, ribonucleoside triphosphates, and a template complementary oligonucleotide that includes a promoter sequence, and optionally may include one or more other oligonucleotides. Variations of transcription associated amplification are well known in the art as disclosed in detail previously (U.S. Pat. Nos. 5,399,491 and 5,554,516, Kacian et al.; U.S. Pat. No. 5,437,990, Burg et al.; PCT Nos. WO 88/01302 and WO 88/10315, Gingeras et al.; U.S. Pat. No. 5,130,238, Malek et al.; U.S. Pat. Nos. 4,868,105 and 5,124,246, Urdea et al.; PCT No. WO 95/03430, Ryder et al.; and US 2006-0046265 A1, Becker et al.). TMA methods (e.g., U.S. Pat. Nos. 5,399,491 and 5,554,516) and single-primer transcription associated amplification method (US 2006-0046265 A1) are embodiments of amplification methods used for detection of P. acnes target sequences as described herein. The person of ordinary skill in the art will appreciate that the disclosed compositions may be used in amplification methods based on extension of oligomer sequences by a polymerase.

“Probe” refers to a nucleic acid oligomer that hybridizes specifically to a target sequence in a nucleic acid, or in an amplified nucleic acid, under conditions that promote hybridization to allow detection of the target sequence or amplified nucleic acid. Detection may either be direct (i.e., a probe hybridized directly to its target sequence) or indirect (i.e., a probe linked to its target via an intermediate molecular structure). A probe's “target sequence” generally refers to a sequence within a larger sequence (e.g., a subset of an amplified sequence) that hybridizes specifically to at least a portion of a probe oligomer by standard base pairing. A probe may comprise target-specific sequences and other sequences that contribute to the three-dimensional conformation of the probe (e.g., described in U.S. Pat. Nos. 5,118,801 and 5,312,728, Lizardi et al.; and U.S. Pat. Nos. 6,849,412, 6,835,542, 6,534,274, 6,361,945, and US 2006-006847 A1, Becker et al.).

Sequence that hybridize to each other may be completely complementary or partially complementary to the intended target sequence by standard nucleic acid base pairing (e.g. G:C, A:T or A:U pairing). By “sufficiently complementary” is meant a contiguous sequence that is capable of hybridizing to another sequence by hydrogen bonding between a series of complementary bases, which may be complementary at each position in the sequence by standard base pairing or may contain one or more residues, including abasic residues, that are not complementary. Sufficiently complementary contiguous bases typically are at least 80%, or at least 90%, complementary to a sequence to which an oligomer is intended to specifically hybridize. Sequences that are “sufficiently complementary” allow stable hybridization of a nucleic acid oligomer with its target sequence under appropriate hybridization conditions, even if the sequences are not completely complementary. Appropriate hybridization conditions are well known in the art, may be predicted based on sequence composition, or can be determined by using routine testing methods (e.g., Sambrook et al., Molecular Cloning, A Laboratory Manual, 2^{nd} ed. (Cold Spring Harbor Laboratory Press, Cold Spring Harbor, N.Y., 1989) at §§ 1.90-1.91, 7.37-7.57, 9.47-9.51 and 11.47-11.57, particularly §§ 9.50-9.51, 11.12-11.13, 11.45-11.47 and 11.55-11.57).

“Sample preparation” refers to any steps or method that treats a sample for subsequent amplification and/or detection of P. acnes nucleic acids present in the sample. Samples may be complex mixtures of components of which the target nucleic acid is a minority component. Sample preparation may include any known method of concentrating components, such as microbes or nucleic acids, from a larger sample volume, such as by filtration of airborne or waterborne particles from a larger volume sample or by isolation of microbes from a sample by using standard microbiology methods. Sample preparation may include physical disruption and/or chemical lysis of cellular components to release intracellular components into a substantially aqueous or organic phase and removal of debris, such as by using filtration, centrifugation or adsorption. Sample preparation may include use of nucleic acid oligonucleotide that selectively or non-specifically capture a target nucleic acid and separate it from other sample components (e.g., as described in U.S. Pat. No. 6,110,678, Weisburg et al., and US 2008-0286775 A1, Becker et al.).

A “capture probe” or “capture oligomer” refers to at least one nucleic acid oligomer that joins a target sequence and an immobilized oligomer by using base pair hybridization. An embodiment of a capture oligomer includes two binding regions: a target sequence-binding region and an immobilized probe-binding region, usually on the same oligomer, although the two regions may be present on different oligonucleotides that are joined by one or more linkers. For example, a first oligomer may include the immobilized probe-binding region and a second oligomer may include the target sequence-binding region, and the two different oligonucleotides are joined by hydrogen bonding with a linker that links the two sequences of the first and second oligonucleotides. Another embodiment of a capture oligomer uses a target-sequence binding region that includes random or non-random poly-GU, poly-GT, or poly U sequences to bind non-specifically to a target nucleic acid and link it to an immobilized probe on a support.

An “immobilized probe” or “immobilized nucleic acid” refers to a nucleic acid that joins, directly or indirectly, a capture oligomer to an immobilized support. One embodiment of an immobilized probe is an oligomer joined to a support that facilitates separation of bound target sequence from unbound material in a sample. Supports may include known materials, such as matrices and particles free in solution, which may be made of nitrocellulose, nylon, glass, polyacrylate, mixed polymers, polystyrene, silane, polypropylene, metal, or other compositions, of which one embodiment is magnetically attractable particles. Supports may be monodisperse magnetic spheres (e.g., uniform size±5%), to which an immobilized probe is joined directly (via covalent linkage, chelation, or ionic interaction), or indirectly (via one or more linkers), where the linkage or interaction between the probe and support is stable during hybridization conditions.

“Separating” or “purifying” means that one or more components of a sample are removed or separated from other sample components. Sample components include target nucleic acids usually in a generally aqueous solution phase, which may also include cellular fragments, proteins, carbohydrates, lipids, and other nucleic acids. Separating or purifying removes at least 70%, or at least 80%, or at least 95% of the target nucleic acid from other sample components.

A “label” refers to a molecular moiety or compound that can be detected or lead to a detectable response or signal. A label may be joined directly or indirectly to a nucleic acid probe. Direct labeling can occur through bonds or interactions that link the label to the probe, including covalent bonds or non-covalent interactions, e.g. hydrogen bonds, hydrophobic and ionic interactions, or formation of chelates or coordination complexes. Indirect labeling can occur through use of a bridging moiety or “linker” such as a binding pair member, an antibody or additional oligomer, which is either directly or indirectly labeled, and which may amplify the detectable signal. Labels include any detectable moiety, such as a radionuclide, ligand (e.g., biotin, avidin), enzyme or enzyme substrate, reactive group, or chromophore (e.g., dye, particle, or bead that imparts detectable color), luminescent compound (e.g., bioluminescent, phosphorescent, or chemiluminescent labels), or fluorophore. Labels may be detectable in a homogeneous assay in which bound labeled probe in a mixture exhibits a detectable change different from that of an unbound labeled probe, e.g., instability or differential degradation properties. A “homogeneous detectable label” can be detected without physically removing bound from unbound forms of the label or labeled probe (e.g., U.S. Pat. Nos. 5,283,174, 5,656,207, and 5,658,737). Labels include chemiluminescent compounds, e.g., acridinium ester (“AE”) compounds that include standard AE and derivatives (e.g., U.S. Pat. Nos. 5,656,207, 5,658,737, and 5,639,604). Synthesis and methods of attaching labels to nucleic acids and detecting labels are well known (e.g., Sambrook et al., Molecular Cloning, A Laboratory Manual, 2nd ed. (Cold Spring Harbor Laboratory Press, Cold Spring Harbor, N.Y., 1989), Chapter 10; U.S. Pat. Nos. 5,658,737, 5,656,207, 5,547,842, 5,283,174, and 4,581,333).

“Consisting essentially of” is used to mean that additional component(s), composition(s) or method step(s) that do not materially change the basic and novel characteristics of the present invention may be included in the compositions or kits or methods of the present invention. Such characteristics include the ability to detect P. acnes nucleic acid in a biological sample at a copy number of about 10⁴ copies of P. acnes. Other characteristics include limited cross-reactivity with other Bacteria or mammalian nucleic acid and targeting 16S or 23 S rRNA. Any component(s), composition(s), or method step(s) that have a material effect on the basic and novel characteristics of the present invention would fall outside of this term.

Disclosed are methods for amplifying and detecting P. acnes nucleic acid, specifically sequences of P. acnes 16S and 23S rRNA or genes encoding 16S and 23S rRNA. The methods include oligonucleotide sequences that specifically recognize target sequences of P. acnes 16S and 23S rRNA or their complementary sequences, or genes encoding 16S and 23S rRNA or their complementary sequences. Such oligonucleotides may be used as amplification oligonucleotides, which may include primers, promoter primers, blocked oligonucleotides, and promoter provider oligonucleotides, whose functions have been described previously (e.g., U.S. Pat. Nos. 5,399,491, 5,554,516 and 5,824,518, Kacian et al.; and US 2006-0046265 A1, Becker et al., and U.S. Pat. Nos. 4,683,195, 4,683,202, and 4,800,159, Mullis et al.). Other oligonucleotides may be used as probes for detecting amplified sequences of P. acnes.

Amplification methods that use TMA amplification include the following steps (described previously in U.S. Pat. Nos. 5,399,491, 5,554,516 and 5,824,518, Kacian et al.). Briefly, the target nucleic acid that contains the sequence to be amplified is provided as single stranded nucleic acid (e.g., ssRNA or ssDNA). Those skilled in the art will appreciate that conventional melting of double stranded nucleic acid (e.g., dsDNA) may be used to provide single-stranded target nucleic acids. A promoter primer binds specifically to the target nucleic acid at its target sequence and a reverse transcriptase (RT) extends the 3′ end of the promoter primer using the target strand as a template to create a cDNA copy of the target sequence strand, resulting in an RNA:DNA duplex. RNase (e.g., RnaseH of the RT) digests the RNA strand of the RNA:DNA duplex and a second primer binds specifically to its target sequence which is located on the cDNA strand downstream from the promoter-primer end. RT synthesizes a new DNA strand by extending the 3′ end of the second primer using the first cDNA template to create a dsDNA that contains a functional promoter sequence. An RNA polymerase specific for the promoter sequence then initiates transcription to produce RNA transcripts that are about 100 to 1000 amplified copies (“amplicons”) of the initial target strand in the reaction. Amplification continues when the second primer binds specifically to its target sequence in each of the amplicons and RT creates a DNA copy from the amplicon RNA template to produce an RNA:DNA duplex. RNase in the reaction mixture digests the amplicon RNA from the RNA:DNA duplex and the promoter primer binds specifically to its complementary sequence in the newly synthesized DNA. RT extends the 3′ end of the promoter primer to create a dsDNA that contains a functional promoter to which the RNA polymerase binds to transcribe additional amplicons that are complementary to the target strand. The autocatalytic cycles of making more amplicon copies repeat during the course of the reaction resulting in about a billion-fold amplification of the target nucleic acid present in the sample. The amplified products may be detected during amplification, i.e., in real-time, or at the end of the amplification reaction by using a probe that binds specifically to a target sequence contained in the amplified products. Detection of a signal resulting from the bound probes indicates the presence of the target nucleic acid in the sample.

One method of transcription associated amplification uses one primer and one or more additional amplification oligonucleotides to amplify nucleic acids in vitro, making transcripts (amplicons) that indicate the presence of the target sequence in a sample (see US2006-0046265 A1 and U.S. Pat. No. 7,374,885, Becker et al.). Briefly, the single primer amplification method uses a primer (or “priming oligomer”), a modified promoter oligomer (or “promoter provider”) that is modified to prevent the initiation of DNA synthesis from its 3′ end (e.g., by including a 3′-blocking moiety) and, optionally, a binding molecule (e.g., a 3′-blocked extender oligomer) to terminate elongation of a cDNA from the target strand. This method synthesizes multiple copies of a target sequence. It includes treating a target RNA that contains a target sequence with primer and a binding molecule, where the primer hybridizes to the 3′ end of the target strand. RT initiates primer extension from the primer's 3′ end to produce a cDNA in a RNA:cDNA duplex with the target strand. When a binding molecule, such as a 3′ blocked extender oligomer, is used in the reaction, it binds to the target strand next to the 5′ end of the target sequence to be amplified. When the primer is extended by DNA polymerase activity of RT to produce cDNA, the 3′ end of the cDNA is determined by the position of the binding molecule because polymerization stops when the primer extension product reaches the binding molecule bound to the target strand. Thus, the 3′ end of the cDNA is complementary to the 5′ end of the target sequence. The RNA:cDNA duplex is separated when RNase (e.g., RNase H of RT) degrades the RNA strand, although those skilled in the art will appreciate that any form of strand separation may be used. Then, the promoter provider oligomer hybridizes to the cDNA near the 3′ end of the cDNA strand. The promoter provider oligomer includes a 5′ promoter sequence for an RNA polymerase and a 3′ region complementary to a sequence in the 3′ sequence of the cDNA. The promoter provider has a modified 3′ end that includes a blocking moiety to prevent initiation of DNA synthesis from the 3′ end of the promoter provider. In the promoter provider:cDNA duplex, the 3′-end of the cDNA is extended by DNA polymerase activity of RT using the promoter oligomer as a template to add a promoter sequence to the cDNA and create a double-stranded functional promoter. RNA polymerase specific for the promoter sequence then binds to the functional promoter and transcribes multiple RNA transcripts complementary to the cDNA, which are amplified RNA identical to the target region sequence that was amplified from the initial target strand. The amplified RNA can cycle through the process again by binding the primer and serving as a template for further cDNA production, ultimately producing many amplified products or amplicons from the initial target nucleic acid present in the sample. Some embodiments of the single primer amplification method do not include the binding molecule and, therefore, the cDNA product made from the primer has an indeterminate 3′ end, but the amplification steps proceed substantially as described above.

Detection of the amplified products may be accomplished by a variety of methods. The nucleic acids may be associated with a surface that results in a physical change, such as a detectable electrical change. Amplified nucleic acids may be detected by concentrating them in or on a matrix and detecting the nucleic acids or dyes associated with them (e.g., an intercalating agent such as ethidium bromide or cyber green), or detecting an increase in dye associated with nucleic acid in solution phase. Other detection methods may use nucleic acid probes that are complementary to a sequence in the amplified product and detecting the presence of the probe:product complex, or by using a complex of probes that may amplify the detectable signal associated with the amplified products (e.g., U.S. Pat. Nos. 5,424,413 and 5,451,503, Hogan et al., and 5,849,481, Urdea et al.). Directly or indirectly labeled probes that specifically associate with the amplified product provide a detectable signal that indicates the presence of the target nucleic acid in the sample. For example, if the target nucleic acid is P. acnes 16S rRNA, the amplified product will contain a sequence in or complementary to a P. acnes 16S rRNA sequence and a probe will bind directly or indirectly to a sequence contained in the amplified product to indicate the presence of P. acnes in the tested sample.

Embodiments of probes that hybridize to the amplified sequences may be DNA or RNA oligonucleotides, or oligonucleotides that contain a combination of DNA and RNA nucleotides, or oligonucleotides synthesized with a modified backbone, e.g., includes one or more 2′-methoxy substituted ribonucleotides. Probes used for detection of the amplified P. acnes rRNA sequences may be unlabeled and detected indirectly (e.g., by binding of another binding partner to a moiety on the probe) or may be labeled with a variety of detectable labels. Label embodiments include compounds that emit a detectable light signal, e.g., fluorophores or luminescent compounds that can be detected in a homogeneous mixture. More than one label, and more than one type of label, may be present on a particular probe, or detection may use a mixture of probes in which each probe is labeled with a compound that produces a detectable signal (e.g., U.S. Pat. Nos. 6,180,340 and 6,350,579, Nelson). Labels may be attached to a probe by various means including covalent linkages, chelation, and ionic interactions. Probes may be linear oligonucleotides that substantially do not form conformations held by intramolecular bonds. Other probes may form conformations by using intramolecular hybridization, e.g. hairpins. Linear probe embodiments include a chemiluminescent compound as the label, e.g. an AE compound.

Hairpin probes may be labeled with any of a variety of different types of interacting labels, where one interacting member is usually attached to the 5′ end of the hairpin probe and the other interacting member is attached to the 3′ end of the hairpin probe. Such interacting members, which may be generally referred to as a reporter dye and a quencher, include a luminescent/quencher pair, luminescent/adduct pair, Forrester energy transfer pair, or a dye dimer. A luminescent/quencher pair may be made up of one or more luminescent labels, such as chemiluminescent or fluorescent labels, and one or more quenchers. In one embodiment, a hairpin probe is labeled at one end with a fluorescent label (“F”) that absorbs light of a particular wavelength or range and emits light another emission wavelength or range and at the other end with a quencher (“Q”) that dampens, partially or completely, signal emitted from the excited F when Q is in proximity with the fluorophore. Such a hairpin probe may be referred to as labeled with a fluorescent/quencher (F/Q) pair. Fluorophores are well known compounds that include, e.g., acridine and its derivatives such as 2-methoxy-6-chloro-9-amino-acridine, coumarin 500, ethidium, fluorescein and its derivatives such as 5 carboxy-fluorescein (FMA), tetrachloro-6-carboxyfluorescein, and 2,7-dimethyl-4,5-dichloro-6-carboxy-fluorescein (JOE), rhodamine and its derivatives such as sulforhodamine 101, N,N,N′,N′-tetramethyl-6-carboxy-rhodamine (TAMRA™), 6-carboxy-X-rhodamine (ROX), HEX, TET, IRD40, IRD41, 5-(2′-aminoethyl)aminoaphthaline-1-sulfonic acid (EDANS™), Texas Red, RedX, eosine and its derivatives, ALEXA dyes such as ALEXA-488, ALEXA-532, ALEXA-546, ALEXA-568 and ALEXA-594, BODIPY dyes such as BODIPY-FL, BODIPY-TMR, BODIPY-TR, BODIPY-630/650 and BODIPY-650-670, cyamine dyes such as Cy3, Cy3.5 and Cy5, oxazine fluorophores such as MR121, lucifer yellow and other dyes that are well known in the art, or fluorescent bases such as 2-aminopurine and pyrrolo-dC (Tyagi et al., 1998, Nature Biotechnol. 16:49-53, Vet et al., 2002, Expert Rev. Mol. Diagn.2(1):77-86, Marti et al, 2006, Nucl. Acids Res. 34(6):50). Quenchers are also well known and include, e.g., 4-(4′-dimethyl-amino-phenylaxo) benzoic acid (DABCYL™), thallium, cesium, p-xylene-bis-pyridinium bromide, and intrinsic properties of nucleotides in nucleic acids, such as deoxyguanosine residues. Different F/Q combinations are well known and many combinations may function together, e.g., DABCYL™ with fluorescein, rhodamine, or EDANS compounds. Other combinations of labels for hairpin probes include a reporter dye, e.g., FAM™, TET™, JOE™, VIC™ combined with a quencher such as TAMRA™ or a non-fluorescent quencher.

One embodiment of a hairpin probe is a “molecular torch” that detects an amplified product to indicate whether a target P. acnes sequence is present in the sample after the amplification step. A molecular torch probe contains a target binding domain to hybridize to the target sequence, if present, resulting in an “open” conformation; a target torch closing domain that hybridizes to the target binding domain in the absence of the target sequence resulting in a “closed” conformation; and a joining region that joins the two domains (e.g., see U.S. Pat. Nos. 6,849,412, 6,835,542, 6,534,274, and 6,361,945, Becker et al.). The target binding domain forms a more stable hybrid with the target sequence than with the target closing domain under the same hybridization conditions so that in the presence of the amplified target sequence, it is in open conformation that produces an amount of signal that is distinguishable from that of the closed conformation. Another hairpin probe embodiment is a “molecular beacon” that includes a label on one arm of the hairpin sequence, a quencher on the other arm, and a loop region joining the two arms (see Tyagi et al., 1998, Nature Biotechnol. 16:49-53, U.S. Pat. Nos. 5,118,801 and 5,312,728, Lizardi et al.). Methods for using such hairpin probes to detect the presence of a target sequence are well known in the art.

One embodiment of a method for detection of P. acnes sequences uses a transcription associated amplification with a hairpin probe, e.g. molecular torch or molecular beacon, which probe may be added before or during amplification, and detection is carried out without subsequent reagent addition. For example, a probe is designed so that the Tm of the hybridized arms of the hairpin probe (e.g., target binding domain:target closing domain complex of a molecular torch) is higher than the amplification reaction temperature to prevent the probe from prematurely binding to amplified target sequences. After an amplification interval, the mixture is heated to open the torch probe arms and allow the target binding domain to hybridize to its target sequence in the amplified product. Then, the solution is cooled to close probes not bound to amplified products, which brings the label/quencher (e.g., F/Q) pair together, allowing detection of signals from probes hybridized to the amplified target sequences in a homogeneous manner. The mixture containing the F/Q labeled hairpin probe is irradiated with the appropriate excitation light and the emission signal is measured.

In other embodiments, the hairpin detection probe is designed so that the amplified products hybridize to the target binding domain of the probe during amplification, resulting in changing the hairpin to its open conformation during amplification, and the amplification reaction mixture is irradiated at intervals to detect the emitted signal from the open probes in real time during amplification.

Preparation of samples for amplification and detection of P. acnes sequences may include methods of separating and/or concentrating organisms contained in a sample from other sample components, e.g., filtration of particulate matter from air, water or other types of samples. Sample preparation may include routine methods of disrupting cells or lysing bacteria to release intracellular contents, including P. acnes 16S or 23S rRNA or genetic sequences encoding 16S or 23S rRNA. Sample preparation before amplification may include an optional step of target capture to specifically or non-specifically separate the target nucleic acids from other sample components. Nonspecific target capture methods may involve selective precipitation of nucleic acids from a substantially aqueous mixture, adherence of nucleic acids to a support that is washed to remove other sample components, other methods of physically separating nucleic acids from a mixture that contains P. acnes nucleic acid and other sample components.

In one embodiment, P. acnes rRNA or genes encoding rRNA are selectively separated from other sample components by specifically hybridizing the P. acnes nucleic acid to a capture oligomer specific for a P. acnes target sequence to form a target sequence:capture probe complex that is separated from sample components by binding the P. acnes target: capture probe complex to an immobilized probe, and separating the P. acnes target:capture probe:immobilized probe complex from the sample, as previously described (U.S. Pat. Nos. 6,110,678, 6,280,952, and 6,534,273, Weisburg et al.). Briefly, the capture probe includes a sequence that specifically binds to the P. acnes target sequence in 16S or 23S rRNA or in a gene encoding 16S or 23S rRNA and also includes a specific binding partner that attaches the capture probe with its bound target sequence to a support, to facilitate separating the target sequence from the sample components. In one embodiment, the specific binding partner of the capture probe is a 3′ “tail” sequence that is not complementary to the P. acnes target sequence but that hybridizes to a complementary sequence on an immobilized probe attached to a support. Any sequence may be used in a tail region, which is generally about 5 to 50 nt long, and other embodiments include a substantially homopolymeric tail of about 10 to 40 nt (e.g., A₁₀ to A₄₀), or about 14 to 33 nt (e.g., A₁₄ to A₃₀ or T₃A₁₄ to T₃A₃₀), that bind to a complementary immobilized sequence (e.g., poly-T) attached to the support, e.g., a matrix or particle. Target capture may occur in a solution phase mixture that contains one or more capture oligonucleotides that hybridize specifically to a P. acnes rRNA or gene target sequence under hybridizing conditions, usually at a temperature higher than the Tm of the tail sequence:immobilized probe sequence duplex. The P. acnes target:capture probe complex is captured by adjusting the hybridization conditions so that the capture probe tail hybridizes to the immobilized probe, and the entire complex on the support is then separated from other sample components. The support with the attached immobilized probe capture probe: P. acnes target sequence may be washed one or more times to further remove other sample components. Other embodiments use a particulate support, such as paramagnetic beads, so that particles with the attached P. acnes target:capture probe:immobilized probe complex may be suspended in a washing solution and retrieved from the washing solution, by using magnetic attraction. To limit the number of handling steps, P. acnes target nucleic acid may be amplified by mixing the target sequence in the complex on the support with amplification oligonucleotides and performing the amplification steps.

Another target capture method uses a capture probe that binds non-specifically to the P. acnes nucleic acid and captures the P. acnes target:capture probe complex to a support, such as by using an immobilized probe or binding pair member on the support. Non-specific capture probes may be poly-U, poly-GU, poly-GT, or random sequences (as described in detail in US 2008-0286775 A1, Becker et al).

Assays for detection of R acnes nucleic acid may optionally include a non-P. acnes internal control (IC) nucleic acid that is amplified and detected in the same assay reaction mixtures by using amplification and detection oligomers specific for the IC sequence. Amplification and detection of a signal from the amplified IC sequence demonstrates that the assay reagents, conditions, and performance of assay steps were properly used in the assay if no signal is obtained for the intended target P. acnes nucleic acid (i.e., samples that test negative for P. acnes). An IC may be used as an internal calibrator for the assay when a quantitative result is desired, i.e., the signal obtained from IC amplification and detection is used to set a parameter used in an algorithm for quantitating the amount of P, acnes nucleic acid in a sample based on the signal obtained for an amplified a P. acnes target sequence. One embodiment of an IC is a randomized sequence that has been derived from a naturally occurring source (e.g., an HIV sequence that has been rearranged randomly). An IC may be an RNA transcript isolated from a naturally occurring source or synthesized in vitro, such as by making transcripts from a cloned randomized sequence such that the number of IC copies in an assay is accurately determined. Primers and probe for the IC target sequence are designed and synthesized by using any well known method provided that the primers and probe function for amplification of the IC target sequence and detection of the amplified IC sequence using substantially the same assay conditions used to amplify and detect the P. acnes target sequence. In embodiments that include a target capture-based purification step, capture of the IC target added to the sample may be included in the assay so that the IC is treated in all of the assay steps similar to the intended P. acnes analyte. Sequences that may be used for amplifying and detecting an internal control include SEQ ID Nos. 205-209.

Amplification and Detection of 16S rRNA Sequences of P. acnes

For amplification and detection of sequences found in the 16S rRNA sequences (which include 16S rRNA and genes encoding 16S rRNA) of P. acnes, oligonucleotides were designed that act as amplification oligomers and detection probes by comparing known sequences of 16S rRNA or gene sequences encoding 16S rRNA and selecting sequences that are common to P. acnes isolates, but may not be completely shared with 16S rRNA sequences of other Propionibacterium species or other bacteria. Sequence comparisons were conducted by using known 16S rRNA sequences (rRNA or genes) of Propionibacterium species (e.g., P. theonii, P. avidum, P. jensenii, P. cyclohexanicum, P. granulosum, P. freudenreichii, P. acidipropionici, and P. propionicus) and of other bacterial species (e.g., Corynebacterium, Fusobacterium, Actinobacterium, Mycobacterium, and Nocardioides). Specific sequences were selected, synthesized in vitro, and the oligomers were characterized by determining the Tm and hybridization characteristics of the P. acnes oligomers with complementary target sequences (synthetic or purified rRNA from bacteria) by using standard laboratory methods. Then, selected oligomer sequences were further tested by making different combinations of amplification oligomers (selected from those shown in Table 1) in amplification reactions with synthetic 16S RNA target sequences or 16S rRNA purified from various Propionibacterium species grown in culture and performing amplification reactions to determine the efficiency of amplification of the 16S rRNA target sequences. The relative efficiencies of different combinations of amplification oligomers were monitored by detecting the amplified products of the amplification reactions, generally by binding a labeled probe (Table 2) to the amplified products and detecting the relative amount of signal that indicated the amount of amplified product made.

Specific oligonucleotides were designed to amplify and detect target sequences in P. acnes 16S rRNA or DNA encoding 16S rRNA, namely within SEQ ID NO: 199, which is based on a P. acnes 16S rRNA gene sequence (contained in GenBank accession number AY642048). Two sets of amplification and detection oligomers were designed that are specific for the 16S rRNA target sequences within a first subset target region, which is SEQ ID NO:200, and the second set of oligomers are specific for the 16S rRNA target sequences within SEQ ID NO. 201.

Embodiments of amplification oligomers for P. acnes 16S rRNA sequences include those shown in Table 1. Amplification oligomers include those that may function as primers, promoter primers, and promoter provider oligomers, with promoter sequences shown in lower case in Table 1. Some embodiments are the target-specific sequence of a promoter primer oligomer listed in Table 1, which optionally may be attached to the 3′ end of any known promoter sequence. Examples of promoter sequences specific for the RNA polymerase of bacteriophage T7 include those of SEQ ID Nos. 73 and 74.

Amplification oligomers may be modified by synthesizing the oligomer with a 3′ blocked end. The blocked oligomers may be used in a single primer transcription associated amplification reaction, i.e., functioning as blocking molecules or promoter provider oligomers. Embodiments of 3′ blocked oligomers include those of SEQ ID Nos. 1-8 and 35-54 where an additional blocked C is added to the 3′ end of the sequence.

TABLE 1
16S rRNA Amplification Oligonucleotides

| | |
|---|---|
| SEQ ID | |
| NO | Sequence |

| | |
|---|---|
| 1 | CGCCGCCAGCGUUCGU |
| 2 | AAGCACGCCGCCAGCGUU |
| 3 | UUAAGCACGCCGCCAGCG |
| 4 | UGUGCAAUAUUCCCCACUGC |
| 5 | AUUCCCCACUGCUGCC |
| 6 | ACUGCUGCCUCCCGUA |
| 7 | GUUCGUCCUGAGCCAGGAU |
| 8 | AGCGUUCGUCCUGAGCC |
| 16 | CCCAAAGTCAAGGGCAGGTTACTC |
| 17 | CCCAAAGTCAAGGGCAGGTTACT |
| 18 | CCCAAAGTCAAGGGCAGGTTAC |
| 19 | GTTATCCCAAAGTCAAGGGCAGGTTAC |
| 20 | CCTGAAGTTATCCCAAAGTCAAGGGCAGGT |
| 21 | CGGTGCTTCTTTACCCATTACCG |
| 22 | CGTAGTTAGCCGGTGCTTCTTTACCCATT |
| 23 | GCACGTAGTTAGCCGGTGCTTGTTTACC |
| 24 | GCCGGTGCTTCTTTACCCATTACCG |
| 25 | GCTTCTTTACCCATTACCGTCACTCACGC |
| 26 | CGTCACTCACGCTTCGTCACAGGC |
| 184 | CTGCTTTTGTGGGGTGCTC |
| 35 | aatttaatacgactcactatagggagaCGAACGCTGGCGGCGTG |
| | CTTAACACATGC |
| 36 | CGAACGCTGGCGGCGTGCTTAACACATGC |
| 37 | aatttaatacgactcactatagggagaACGCTGGCGGCGTGCTT |
| | AACAGATGC |
| 38 | ACGCTGGCGGCGTGCTTAACACATGC |
| 39 | aatttaatacgactcactatagggagaCGGCGTGCTTAACACAT |
| | GCAAGTCG |
| 40 | CGGCGTGCTTAACACATGCAAGTCG |
| 41 | aatttaatacgactcactatngggagaCGTGCTTAACACATGCA |
| | AGTCGAACGG |
| 42 | CGTGCTTAACACATGCAAGTCGAACGG |
| 43 | aatttaatacgactcactatagggagaCTTAACACATGCAAGTC |
| | GAACGGAAAGG |
| 44 | CTTAACACATGCAAGTCGAACGGAAAGG |
| 45 | aatttaatacgactcactatagggagaGCACAATGGGCGGAAGC |
| | CTGATGCAGCAACG |
| 46 | GCACAATGGGCGGAAGCCTGATGCAGCAACG |
| 47 | aatttaatacgactcactatagggagaGCACAATGGGCGGAAGC |
| | CTGATGCAGC |
| 48 | GCACAATGGGCGGAAGCCTGATGCAGC |
| 49 | aatttaatacgactcactatagggagaGCACAATGGGCGGAAGC |
| | CTGATGC |
| 50 | GCACAATGGGCGGAAGCCTGATGC |
| 51 | aatttaatacgactcactatagggagaGGAATATTGCACAATGG |
| | GCGGAAGC |
| 52 | GGAATATTGCACAATGGGCGGAAGC |
| 53 | aatttaatacgactcactatagggagaGCAGTGGGGAATATTGC |
| | ACAATGGGCG |
| 54 | GCAGTGGGGAATATTGCACAATGGGCG |
| 73 | aatttaatacgactcactatagggaga |
| 74 | agagggatatcactcagcataatttaa |

Embodiments of detection probe oligomers for detection of amplified products of 16S rRNA sequences or genes encoding 16S rRNA are shown in Table 2. Detection probe embodiments include oligomers that can form hairpin configurations by intramolecular hybridization of the probe sequence, which include those of SEQ ID Nos. 75-98. For the sequences listed in Table 2, the lowercase letters indicate the sequences that hold a hairpin oligomer in the closed configuration. Embodiments of the hairpin probe oligomers were synthesized with a fluorescent label attached at one end of the sequence and a quencher compound attached at the other end of the sequence. Embodiments of hairpin probes may be labeled with a 5′ fluorophore and a 3′ quencher, for example a 51 fluorescein label and a 3′ DABCYL quencher. Some embodiments of hairpin oligomers also include a non-nucleotide linker moiety at selected positions within the sequence. Examples of such embodiments include those that include an abasic 9-carbon (“C9”) linker between residues 16 and 17 of SEQ ID Nos.: 75-84, between residues 17 and 18 of SEQ ID Nos.: 85-88, between residues 18 and 19 of SEQ ID Nos.: 89-92, between residues 19 and 20 of SEQ ID Nos.: 93 and 94, between residues 20 and 21 of SEQ ID Nos.: 95 and 96, and between residues 21 and 22 of SEQ ID Nos.: 97 and 98.

Another detection probe embodiment is a linear detection probe oligomers labeled with a chemiluminescent AE compound which is attached to the probe sequence via a linker (substantially as described in U.S. Pat. Nos. 5,585,481 and 5,639,604, particularly at column 10, line 6 to column 11, line 3, and in Example 8). Examples of labeling positions are a central region of the probe oligomer and near a region of A:T base pairing, at a 3′ or 5′ terminus of the oligomer, and at or near a mismatch site with a known sequence that is not the desired target sequence.

The detection probes may be used with helper probes that are unlabeled and facilitate binding of the labeled probe to its target (see U.S. Pat. No. 5,030,557, Hogan et al.).

TABLE 2
16S rRNA Probes

| | |
|---|---|
| SEQ ID NO | Sequence |

| | |
|---|---|
| 75 | CGAGCACCCCACAAAAGCucg |
| 76 | CGAGCACCCCACAAAAGC |
| 77 | CGAGCACCCCACAAAAGgcucg |
| 78 | CGAGCACCCCACAAAAG |
| 79 | GCACCCCACAAAAGCAGGugc |
| 80 | GCACCCCACAAAAGCAGG |
| 81 | CCGUCACUCACGCUUCGacgg |
| 82 | CCGUCACUCACGCUUCG |
| 83 | GCGGUUUACAACCCGAAccgc |
| 84 | GCGGUUUACAACCCGAA |
| 85 | GCCACUCGAGCACCCCAuggcg |
| 86 | GCCACUCGAGCACCCCA |
| 87 | CCCGAAGGCCGUCAUCCucggg |
| 88 | CCCGAAGGCCGUCAUCC |
| 89 | CGCCACUCGAGCACCCCAuggcg |
| 90 | CGCCACUCGAGCACCCCA |
| 91 | cCGUCACAGGCGAAAGCGGacgg |
| 92 | CGUCACAGGCGAAAGCGG |
| 93 | CGCCACUCGAGCACCCCACuggcg |
| 94 | CGCCACUCGAGCACCCCAC |
| 95 | CGCCACUCGAGCACCCCACAuggcg |
| 96 | CGCCACUCGAGCACCCCACA |
| 97 | CACGCUUCGUCACAGGCGAAAGCgug |
| 98 | CACGCUUCGUCACAGGCGAAAGC |
| 181 | cCAGGGCCUUUCCGUUCGccugg |
| 182 | CAGGGCCUUUCCGUUCG |

Capture probe oligomers may be used in sample preparation to separate P. acnes 16S rRNA target nucleic acids from other sample components include those that contain the target-specific sequences of SEQ ID Nos. 120 and 122. Embodiments of the capture probes include a 3′ tail region covalently attached to the target-specific sequence to serve as a binding partner that binds a hybridization complex made up of the target nucleic acid and the capture probe to an immobilized probe on a support. Embodiments of capture probes that include the target-specific sequences of SEQ ID Nos. 128 and 130, further include 3′ tail regions made up of substantially homopolymeric sequences, such as dT₃A₃₀ polymers (SEQ ID Nos. 127 and 129). Examples of target capture probes are listed in Table 3.

TABLE 3
Target Capture Probes for P. acnes 16S rRNA

| | |
|---|---|
| 127 | GCCUUGGUAAGCCACUAtttaaaaaaaaaaaaaaaaaaaaaaaaaaa |
| | aaa |
| 128 | GCCUUGGUAAGCCACUA |
| 129 | GCUGAUAAGCCGCGAGUtttaaaaaaaaaaaaaaaaaaaaaaaaaaa |
| | aaa |
| 130 | GCUGAUAAGCCGCGAGU |

Amplification and Detection of 23S rRNA Sequences of P. acnes

For amplification and detection of P. acnes sequences found in 23S rRNA, which include 23S rRNA sequence or DNA encoding 23S rRNA, oligonucleotides were designed that act as amplification oligomers and detection probes by comparing known sequences of 23S rRNA or gene sequence encoding 23S rRNA and selecting sequences that are common to P. acnes isolates, but may not be completely shared with 23S rRNA sequences of other Propionibacterium species or other bacteria. Sequence comparisons were conducted by using known 23S rRNA sequences (RNA or genes) of Propionibacterium species (P. granulosum, P. avidum, P. thoenii, P. freudenreichii, P. jensenii, and P. acidipropionici) and of other bacterial species (Campylobacter sp., Mycobacterium sp., and Helicobacter sp.). Specific sequences were selected, synthesized in vitro, and the oligomers were characterized by determining the hybridization characteristics of the P. acnes oligonucleotides with complementary target sequences (synthetic or purified rRNA from bacteria) by using standard laboratory methods. Then, selected oligonucleotide sequences were further tested by making different combinations of amplification oligomers (selected from those shown in Table 4) in amplification reactions with synthetic 23S RNA target sequences or 23S rRNA purified from various Propionibacterium species grown in culture and performing amplification reactions to determine the efficiency of amplification of the 23S rRNA target sequences. The relative efficiencies of different combinations of amplification oligomers were monitored by detecting the amplified products of the amplification reactions, generally by binding a labeled probe to the amplified products and detecting the relative amount of signal that indicated the amount of amplified product made.

Specific oligonucleotides were designed to amplify and detect target sequences in P. acnes 23S rRNA or DNA encoding 23S rRNA, namely within SEQ ID NO:202, which is based on a P, acnes 23S rRNA gene sequence (contained in GenBank accession number AE017283). Two sets of amplification and detection oligomers were designed that are specific for the 23S rRNA target sequences within a first subset target region, which is SEQ ID NO:203, and the second set of oligomers are specific for the 23S rRNA target sequences within SEQ ID NO. 204.

Embodiments of amplification oligomers for P. acnes 23S rRNA sequences include those shown in Table 4, in which lower case letters are used for the promoter sequences in promoter primers. Other embodiments of promoter primers may include the target-specific sequence of an oligomer listed in Table 4 attached to the 3′ end of any of a variety of promoter sequences.

TABLE 4

Amplification Oligonucleotides
for 23S rRNA Target Sequences

| | |
|---|---|
| SEQ ID | |
| NO | Sequence |

| | |
|---|---|
| 9 | ACCCAACCACACAGGTTGCC |
| 10 | CTACCCAACCACACAGGTTG |
| 11 | CACTGACACGACGCTCCCCT |
| 12 | CCTACCCAACCACACAGGTT |
| 13 | GCTCCCCTACCCAACCACAC |
| 14 | TAAGCCCCCTAGACCATCCA |
| 15 | TGCTCTACCTCCAACAAGAAC |
| 188 | ACUCCACAUCCUUUCCC |
| 27 | GGATTCCCACCCGTCACA |
| 28 | GGATTCCCACCCGTCACAC |
| 29 | GGATTCCCACCCGTCACACG |
| 30 | GATTCCCACCCGTCACACG |
| 31 | GATTCCCACCCGTCACAC |
| 32 | ATTCCCACCCGTCACAC |
| 33 | CCACATCCTTTCCCACTGAGTC |
| 34 | CTTATCCCCCGCCGTCTCACTGCC |
| 185 | GCTTCACCACTGACACGA |
| 55 | aatttaatacgactcactatagggagaTGGGTAGGGGAGCGTCG |
| | TGTCAGT |
| 56 | TGGGTAGGGGAGCGTCGTGTCAGT |
| 57 | aatttaatacgactcactatagggagaTGGGTAGGGGAGCGTCG |
| | TGTCAGTG |
| 58 | TGGGTAGGGGAGCGTCGTGTCAGTG |
| 59 | aatttaatacgactcactatagggagaCAGTGGTGAAGCTGCCG |
| | GGTGACT |
| 60 | CAGTGGTGAAGCTGCCGGGTGACT |
| 61 | aatttaatacgactcactatagggagaCAGTGGTGAAGCTGCCG |
| | GGTGACTG |
| 62 | CAGTGGTGAAGCTGCCGGGTGACTG |
| 63 | aatttaatacgactcactatagggagaGTAGGGGAGCGTCGTGT |
| | CAGTG |
| 64 | GTAGGGGAGCGTCGTGTCAGTG |
| 65 | aatttaatacgactcactatagggagaGGAGCGTCGTGTCAGTG |
| | GTGAAGCTG |
| 66 | GGAGCGTCGTGTCAGTGGTGAAGCTG |
| 67 | aatttaatacgactcactatagggagaGCTTATCGGCTTACCGA |
| | AATCAGCCAAAC |
| 68 | GCTTATCGGCTTACCGAAATCAGCCAAAC |
| 69 | aatttaatacgactcactatagggagaGCTTATCGGCTTACCGA |
| | AATCAGCC |
| 70 | GCTTATCGGCTTACCGAAATCAGCC |
| 71 | aatttaatacgactcactatagggagaGAGCACTGGATGGTCTA |
| | GGGGGCTTATCG |
| 72 | GAGCACTGGATGGTCTAGGGGGCTTATCG |
| 186 | aatttaatacgactatagggagaGGAGTTGCGTAGACAACCAGG |
| 187 | GGAGTTGCGTAGACAACCAGG |

Embodiments of detection probe oligomers for amplified products of 23S rRNA sequences or genes encoding 23S rRNA are shown in Table 5. Detection probe embodiments are oligomers that can form hairpin configurations by intramolecular hybridization of the probe sequence, which include those of SEQ ID Nos. 99-126 and 183. Embodiments of the detection probes include a 5′ fluorophore (e.g., fluorescein), a 3′ quencher (e.g., DABCYL), and an abasic moiety (e.g., C9) between residues 5 and 6 of SEQ ID Nos. 121-126, or between residues 16 and 17 of SEQ ID Nos. 99-100, or between residues 17 and 18 of SEQ ID Nos. 103-106, or between residues 18 and 19 of SEQ ID Nos. 111-112, or between residues 19 and 20 of SEQ ID Nos. 107-110 and 113-114, or between residues 20 and 21 of SEQ ID Nos. 101-102 and 113-116. Another embodiment includes detection probes labeled with an AE attached to the oligomer by a non-nucleotide linker.

TABLE 5
23S rRNA Probes

| | |
|---|---|
| SEQ ID NO | Sequence |

| | |
|---|---|
| 99 | CUCGCAUCAACUCCACgcgAg |
| 100 | CUCGCAUCAACUCCAC |
| 101 | GCCUGCAUUCUCACUCGCAUCAggc |
| 102 | GCCUGCAUUCUCACUCGCAUCA |
| 103 | ccCACUCGCAUCAACUCguggg |
| 104 | CACUCGCAUCAACUC |
| 105 | CUCGCAUCAACUCCACCgcgag |
| 106 | CUCGCAUCAACUCCACC |
| 107 | CUCGCAUCAACUCCACCACgcgag |
| 108 | CUCGCAUCAACUCCACCAC |
| 109 | cgCUCACUCGCAUCAACUCgagcg |
| 110 | CUCACUCGCAUCAACUC |
| 111 | gCUCACUCGCAUCAACUCugagc |
| 112 | CUCACUCGCAUCAACUC |
| 113 | gCUCACUCGCAUCAACUCCugagc |
| 114 | CUCACUCGCAUCAACUCC |
| 115 | gCUCACUCGCAUCAACUCCAugagc |
| 116 | CUCACUCGCAUCAACUCC |
| 117 | CGCUACACUUGCCAGCAUUCagcg |
| 118 | CGCUACACUUGCCAGCAUUC |
| 119 | CUGCCACGCUACACUUGCCAGggcag |
| 120 | CUGCCACGCUACACUUGCCAG |
| 121 | gguggCACUCGCAUCAACUCCACC |
| 122 | CACUCGCAUCAACUCCACC |
| 123 | gguggCUCGCAUCAACUCCACC |
| 124 | CUCGCAUCAACUCCACC |
| 125 | gguggACUCGCAUCAACUCCACC |
| 126 | ACUCGCAUCAACUCCACC |
| 183 | CCGCUACAUUAUCGGCGagcgg |

Embodiments of capture probe oligomers for use in sample preparation to separate P. acnes 23S rRNA target nucleic acids from other sample components include those that contain a target-specific sequence that hybridizes to a 23S rRNA sequence or gene encoding 23S rRNA, such as the target-specific sequence of SEQ ID Nos. 132 and 134. Embodiments of capture probes include a 3′ tail region covalently attached to the target-specific sequence to serve as a binding partner that binds the hybridization complex made up of the target nucleic acid and the capture probe to an immobilized probe on a support. Embodiment of the covalently linked 3′ tail region is a substantially homopolymeric sequence, such as dT₃A₃₀ linked to the 3′ end as shown in SEQ ID Nos. 131 and 133. Sequences of target capture probe embodiments are in Table 6.

TABLE 6
Target Capture Probes for P. acnes 23S rRNA

| | |
|---|---|
| 131 | CAAACUUGACCCUGGAACCCUUGGtttaaaaaaaaaaaaaaaaaaaa |
| | aaaaaaaaaa |
| 132 | CAAACUUGACCCUGGAACCCUUGG |
| 133 | CUUAGGACCCGACUCACCCAGGGCAGACAtttaaaaaaaaaaaaaaa |
| | aaaaaaaaaaaaaaa |
| 134 | CUUAGGACCCGACUCACCCAGGGCAGACA |

### Example 1

### Assay Reagents and Protocols

The experiments described in the examples that follow may use one or more of the following reagents, although those skilled in the art will appreciate that other reagents may be substituted for those described here. Probe Matrix Lysis Reagent contained 0.1% (w/v) LLS, 20 mM Lithium Succinate, and 1 mM EDTA. Lysis Reagent contained 1% (w/v) LLS, 100 mM Tris, 2.5 mM succinic acid, 10 mM EDTA, and 500 mM lithium chloride (LiCl) at pH 6.5-7.5. Target Capture Reagent contained 300 mM HEPES, 1.88 M lithium chloride, 100 mm EDTA, at pH 6.4, and 250 μg/ml of paramagnetic particles (0.7-1.05μ particles, SERA-MAG™ MG-CM, Seradyn, Inc., Indianapolis, Ind.) with (dT)₁₄ oligomers covalently bound thereto. Wash Solution used in target capture contained 10 mM HEPES, 150 mM NaCl, 1 mM EDTA, and 0.1% (w/v) sodium dodecyl sulfate, at pH 7.5. Amplification Reagent was mixed with other reaction components produce a solution containing 50 mM HEPES, 33 mM KCl, 30 mM MgCl₂, 0.5 mM of each dNTP (dATP, dCTP, dGTP, dTTP), 10 mM ATP, 2 mM CTP, 12.7 mM UTP, 2 mM GTP, at pH 7.7. Amplification oligonucleotides (primers, promoter primers, blocker oligonucleotides, promoter provider oligonucleotides), and optionally probes, may be added to the reaction mixture in the amplification reagent or separate from the amplification reagent. Enzyme Reagent contained 360 RTU/μl of Moloney murine leukemia virus (MMLV) reverse transcriptase (RT) and about 80 PU/μl of T7 RNA polymerase, 75 mM HEPES, 120 mM KCl, 10% TRITON® X-100, 160 mM N-acetyl-L-cysteine, and 1 mM EDTA at pH 7.0, where 1 RTU of RT incorporates 1 nmol of dTTP in 20 min at 37° C., and 1 PU of T7 RNA polymerase produces 5 nmol of RNA transcript in 20 min at 37° C. Hybridization Buffer contained 0.6M LiCl, 1% lithium lauryl sulfate, 10 mM of EDTA and 10 mM EDTA, 50 mM succinic acid, 115 mM lithium hydroxide, pH 4.7.

The transcription mediated amplification (TMA) reactions use substantially the procedures as disclosed in detail previously U.S. Pat. Nos. 5,399,491 and 5,554,516 (Kacian et al.). Single primer transcription associated amplification use substantially the procedures disclosed in detail in US 2006-0046265 A1 and U.S. Pat. No. 7,374,885 (Becker et al). The TAG amplification method used procedures substantially as disclosed in US 2007-0281317 A1 (Becker et al.). The use and detection of signal from AE-labeled probes to detect hybridization complexes with target sequences use the procedures substantially as disclosed in detail previously (U.S. Pat. Nos. 5,283,174 and 5,656,744, Arnold et al., and U.S. Pat. No. 5,658,737, Nelson et al.). The methods for using hairpin probes are well known, and include those already disclosed in detail in U.S. Pat. Nos. 6,849,412, 6,835,542, 6,534,274, and 6,361,945, (Becker et al.).

By using various combinations of amplification oligonucleotides and labeled detection probes to provide a detectable signal, P. acnes 16S rRNA and 23S rRNA sequences were specifically detected in samples containing the appropriate target nucleic acids. The following examples illustrate some of the preferred embodiments for these methods and compositions for detection of P. acnes target sequences.

### Example 2

### P. acnes Culture and Lysis

P. acnes were grown on reinforced clostridial agar (RCA) at 37° C. under anaerobic conditions for three to four days. Colonies were selected and further grown in degassed reinforced clostridial broth at 37° C. under anaerobic conditions for 24-48 hours. Bacteria (approximately 2×10⁷ CFU) were pelleted and resuspended in 1 mL of Probe Matrix Lysis Reagent for 15-60 minutes at 80 or 100° C. The bacteria were detected using methods and reagents substantially as are described in detail previously (Arnold et al., 1989, Clin. Chem., vol. 35, pp. 1588-94). Briefly, after lysing the bacteria, 1 mL of 2× Hybridization Buffer was added to the Probe Matrix Lysis Reagent. Hybridization Buffer contained nucleic acid probes specific for rRNA sequences conserved among bacterial species (see U.S. Pat. Nos. 4,851,330, 5,567,587, 5,601,984, 5,688,645, 5,714,324, and 5,723,579, Kohne, and 5,679,520, Hogan et al.) and the probes were labeled with acridinium ester (AE) (see U.S. Pat. Nos. 4,946,958, Campbell et al.; and 5,185,439, 5,585,481 5,656,744, and 5,696,251, Arnold et al.). Non-hybridized probes were deactivated followed by activation of the AE and detection on a luminometer (see U.S. Pat. Nos. 5,238,174 and 5,639,604, Arnold et al.). The amount of nucleic acid released was measured in relative light units (RLU). Table 7 shows the RLU signals over a course of time for the two different incubation temperatures. An alternative to using temperature controlled equipment, e.g. a heat block, bacteria can be lysed using a microwave oven. For example, microwaving at 30% power for 15 minutes may provide similar results as incubating at 95° C. for 60 minutes. This reduces lysis time, however, due to microwave oven power differences, the power level and time may need to be adjusted.

TABLE 7
Lysis Comparison Between 80° C. and 100° C.

| Temperature | 15 minutes | 30 minutes | 45 minutes | 60 minutes |
|---|---|---|---|---|
| 80° C. | 30,834 | 65,154 | 124,729 | 156,817 |
| 100° C. | 69,578 | 186,024 | 244,604 | 361,665 |

In another embodiment, P. acnes bacteria may be filtered from a liquid using a filter system (e.g. MILLIPORE® Microfil S system). P. acnes were dispersed into 0.9% saline and passed through a Microfil S system. The filter was removed and placed in a container with 1 mL of Probe Matrix Lysis Reagent The container was incubated at 100° C. for 30-60 minutes followed by the addition of 1 mL of 2× Hybridization Buffer. Results in Table 8 show the RLU signals for pelleted bacteria and filtered bacteria.

TABLE 8
Lysis Comparison Between Pelleted and Filtered Bacteria

| | 0 minutes | 15 minutes | 30 minutes | 45 minutes | 60 minutes | 90 minutes |
|---|---|---|---|---|---|---|
| Pelleted | 8,525 RLU | 141,692 | 270,732 | 302,359 | 395,197 | 428,571 |
| Filtered | 16,302 RLU | 159,674 | 201,704 | 260,828 | 338,774 | 333,503 |

In another embodiment, P. acnes can be lysed using known enzymes, (e.g. lysozyme, mutanolysin, and protein K), alone or in combination. For example, P. acnes bacteria were incubated in 500 ul TES buffer (30 mM Tris, pH 8.0; 5 mM EDTA; 50 mM NaCl) plus lysozyme for 30 minutes at 37° C. An additional 500 ul of TES buffer plus 0.5% LDS, 1% Triton X-100 and mutanolysin or proteinase K was added and incubated an additional 30 minutes at 37° C. followed by the addition of 1 mL of 2× Hybridization Buffer. A comparison of Probe Matrix Lysis Reagent and protease digestion is shown in Table 9.

TABLE 9
Lysis Comparison Between Heat and Enzyme

RLU

| | |
|---|---|
| Negative control (Hybridization Reagent at room | 1,644 |
| temperature) | |
| Probe Matrix Lysis buffer 100° C. for 60 minutes | 399,713 |
| Lysozyme and mutanolysin in TES buffers | 865,296 |
| Lysozyme and proteinase K in TES buffers | 170,466 |

### Example 3

### Design and Initial Testing of P. acnes 16S Amplification Oligomer Sets

Amplification and detection oligonucleotides were designed to amplify and detect P. acnes 16S rRNA. The designs were based on a P. acnes 16S rRNA gene sequence (GenBank accession number AY642048.1 and GI number 50082571). Two sets of amplification and detection oligomers were designed to be specific for target sequences within the first 500 bases of GI number 50082571 (SEQ ID NO. 199), in which the first set of oligomers are specific for target sequences contained in bases 1-200 (SEQ ID NO. 200) and the second set of oligomers are specific for target sequences contained in bases 325-500 (SEQ ID NO. 201). The P. acnes 16S rRNA amplification and detection oligomers are SEQ ID Nos. 1-8, 16-26, 35-54, 75-98, 135-160, 181-182, 184, 189-193, 210, and 211-216.

Known amounts of P. acnes 16S rRNA sequences were amplified using real time single primer transcription associated amplification, (substantially as described in detail in US20060046265 A1, Becker et al.), using combinations of the oligonucleotides in Table 1 and Table 2. Oligomer combinations were tested using 0 and 105 copies of P. acnes 16S rRNA. The reaction mixtures containing the amplification oligonucleotides, target and amplification reagents (but not enzymes) were covered to prevent evaporation, incubated 5 min at 95° C., then 2 min at 42° C., then enzymes were added, the reactions were mixed and incubated for 30 min at 42° C., measuring fluorescence at regular time intervals (i.e., every 30 sec) during the amplification reaction after enzyme addition. The reaction mixture was monitored using a real time fluorescent detection system (e.g. Bio-Rad Laboratory's Opticon™ or Chromo4™, or a FluoDia® T70 instrument). Real time algorithms are well known in the art (e.g., see Biochem. Biophys. Res. Commun. 2002 Jun. 7; 294(2):347-53 and Nucleic Acids Res. 2004 32(22):e178). Negative controls, (reactions performed in the same manner using zero copies of P. acnes 16S rRNA), were tested to determine background fluorescent signal levels. Oligomer combinations were evaluated on signal emergence time, maximum relative fluorescent units (RFU) signal, slope of the sigmoid curve, and background signal. Assay oligomer combinations that produced a fluorescent curve at 10⁵ copies of P. acnes 16S rRNA and had less than 0.1 relative fluorescent units (RFU) in the negative controls were selected for further testing.

The following assay oligomer combinations were further tested for sensitivity: (1) SEQ ID Nos. 8, 20, 35 and 75; (2) SEQ ID Nos. 7, 20, 35, and 79; (3) SEQ ID Nos. 7, 20, 35 and 77; (4) SEQ ID Nos. 1, 16, 37, and 75; (5) SEQ ID Nos. 1, 18, 37, and 75; (6) SEQ ID Nos. 3, 20, 41, and 75; (7) SEQ ID Nos. 8, 19, 37, and 79; (8) SEQ ID Nos. 1, 16, 37, and 79; (9) SEQ ID Nos. 3, 19, 37, and 95; (10) SEQ ID Nos. 4, 22, 47, and 81; and (11) SEQ ID Nos. 4, 23, 51, and 81. Each combination was tested at 0, 10³, 10⁴, and 10⁵ copies of P. acnes 16S rRNA. These results were used to identify assay oligomer combinations that displayed emergence times around 20 minutes for reactions that contained 10⁵ copies of P. acnes 16S rRNA, with maximum signals from 0.98 to 1.5 RFU, and background signal levels at less than 0.2 RFU. The results of these tests showed that selected assay oligomer combinations amplified as few as 10⁴ copies of P. acnes 16S rRNA. Results obtained by using the 16S amplification and detection oligomer combinations of combination 1 (SEQ ID Nos. 8, 20, 35 and 75), combination 2 (SEQ ID Nos. 7, 20, 35, and 79), and combination 3 (SEQ ID Nos. 7, 20, 35 and 77) are shown in Table 10, reported as emergence of the signal (TTime) and average RFU (“AveRange” or mean of detected RFU for replicate samples in the same condition, averaged following subtraction of the minimum detected RFU for a test result from the maximum detected RFU from the same test result).

TABLE 10
16S rRNA Amplification and Detection Oligomer
Combinations for P. acnes

| Amplification | Copies | AveRange | TTime |
|---|---|---|---|
| Oligonucleotides | rRNA | (RFU) | (minutes) |
| Combination 1 | 0 | 0.208 | 35.1 |
| | 10³ | 0.527 | 33.0 |
| | 10⁴ | 1.176 | 28.2 |
| | 10⁵ | 1.452 | 23.1 |
| Combination 2 | 0 | 0.116 | 28.0 |
| | 10³ | 0.215 | 27.9 |
| | 10⁴ | 0.771 | 26.6 |
| | 10⁵ | 0.984 | 22.2 |
| Combination 3 | 0 | 0.085 | 42.6 |
| | 10³ | 0.362 | 31.9 |
| | 10⁴ | 0.962 | 27.7 |
| | 10⁵ | 1.248 | 22.3 |

Amplification and detection oligomer combinations 1 (SEQ ID Nos. 8, 20, 35 and 75), 2 (SEQ ID Nos. 7, 20, 35, and 79) and 3 (SEQ ID Nos. 7, 20, 35, and 77) were tested for cross reactivity against the following challenge organisms: P. granulosum, P. cyclohexanicum, P. freudenreichii, aclinomyces israeli, P. avidum and P. theonii. Each oligomer combination was tested for each challenge organism using single primer transcription associated amplification. The oligomer combinations 1, 2 and 3 were tested using 104 CFU for P. granulosum, P. cyclohexanicum, P. freudenreichii and A. israelii, and 10² CFU for P. avidum and P. theonii. Amplification and detection oligomer combinations did not amplify or detect any of the challenge organisms. Data from the cross reactivity test is shown in Table 11 reported as described above for each of the oligomer combinations.

TABLE 11
Cross Reactivity for the 16S rRNA Assay Oligomer Combinations

| Amplification | Challenge | AveRange | TTime |
|---|---|---|---|
| Oligonucleotides | Organism | (RFU) | (minutes) |
| Combination 1 | none | 0.294 | 33.8 |
| | P. avidum | 0.068 | No signal |
| | P. cyclohexanicum | 0.177 | 36.5 |
| | P. freudenreichii | 0.063 | No signal |
| | P. granulosum | 0.064 | No signal |
| | P. israellii | 0.062 | No signal |
| | P. theonii | 0.055 | No signal |
| Combination 2 | none | 0.134 | 26.5 |
| | P. avidum | 0.035 | No signal |
| | P. cyclohexanicum | 0.093 | 14.7 |
| | P. freudenreichii | 0.041 | No signal |
| | P. granulosum | 0.037 | No signal |
| | P. israellii | 0.036 | No signal |
| | P. theonii | 0.049 | No signal |
| Combination 3 | none | 0.134 | 26.5 |
| | P. avidum | 0.035 | No signal |
| | P. cyclohexanicum | 0.093 | 14.7 |
| | P. freudenreichii | 0.041 | No signal |
| | P. granulosum | 0.037 | No signal |
| | P. israellii | 0.036 | No signal |
| | P. theonii | 0.049 | No signal |

The amplification and detection oligomer combinations were further tested ensure that challenge organisms (i.e., related non-P. acnes species), did not inhibit amplification of P. acnes 16S rRNA. Three challenge organisms, P. cyclohexanicum, P. avidum and P. theonii, were chosen because P. avidum exhibits the highest degree of homology to P. acnes, whereas P. theonii and P. cyclohexanicum exhibited the strongest affects on the fluorescent curves in the initial cross reactivity studies. Zero or 106 copies of P. acnes 16S rRNA were tested with 10⁴ copies of P. Cyclohexanicum or 10² copies of P. avidum or P. theonii. The tests were repeated using 0 or 10⁴ CFU P. acnes lysate and 10⁴ copies of P. cyclohexanicum, P. avidum or P. theonii. Samples without P. acnes showed no amplification where as samples with P. acnes showed amplification. The presence of other bacteria reduced the background signal and the maximum RFU's, however, the emergence time was not affected. Data from the tests is show in Table 12.

TABLE 12
Specific Amplification of P. acnes 16S rRNA in the
Presence of Challenge Organisms

| Amplification | P. acnes | Challenge | AveRange | TTime |
|---|---|---|---|---|
| Oligomer | rRNA | Organism | (RFU) | (minutes) |
| Combination 1 | 0 | none | 0.657 | 35.9 |
| | 10⁶ | none | 1.894 | 22.4 |
| | 0 | P. avidum | 0.067 | No signal |
| | 10⁶ | P. avidum | 1.393 | 23.2 |
| | 0 | P. cyclohexanicum | 0.345 | 38.1 |
| | 10⁶ | P. cyclohexanicum | 1.815 | 23.0 |
| | 0 | P. theonii | 0.060 | No signal |
| | 10⁶ | P. theonii | 0.920 | 22.3 |
| Combination 2 | 0 | none | 0.249 | 27.8 |
| | 10⁶ | none | 1.316 | 19.0 |
| | 0 | P. avidum | 0.033 | No signal |
| | 10⁶ | P. avidum | 0.909 | 19.4 |
| | 0 | P. cyclohexanicum | 0.103 | 23.7 |
| | 10⁶ | P. cyclohexanicum | 1.182 | 19.7 |
| | 0 | P. theonii | 0.049 | 0.9 |
| | 10⁶ | P. theonii | 1.040 | 19.4 |
| Combination 3 | 0 | none | 0.371 | 35.7 |
| | 10⁶ | none | 1.752 | 21.0 |
| | 0 | P. avidum | 0.048 | No signal |
| | 10⁶ | P. avidum | 1.407 | 21.5 |
| | 0 | P. cyclohexanicum | 0.203 | 38.2 |
| | 10⁶ | P. cyclohexanicum | 1.633 | 21.5 |
| | 0 | P. theonii | 0.047 | No signal |
| | 10⁶ | P. theonii | 1.380 | 21.1 |

### Example 4

### Design and Testing of P. acnes 16S rRNA Target Capture Oligomers

Target capture is used to purify and prepare nucleic acid samples for subsequent amplification (see U.S. Pat. Nos. 6,110,678, 6,280,952, and 6,534,273, Weisburg et al.). Briefly, samples were prepared containing known amounts of target nucleic acid in Lysis Reagent. Samples were mixed with a target capture oligomer that contained the target-specific sequence with a covalently attached dT₃A₃₀ tail, and polydT-magnetic particles. The mixtures were incubated for 30 min at 60° C., and then for 30 min at room temperature to form hybridization complexes that captured the target RNA onto the magnetic particles. Magnetic particles were separated from the solution by applying a magnetic field to the outside of the container and transferring the magnetic particles to another solution. The magnetic particles with the target sequence were washed once with wash solution. The magnetic particles with the target nucleic acid were resuspended in amplification reagent, ready for subsequent amplification. Target capture can be performed manually or with an automated device (e.g. Target Capture System (Gen-Probe Incorporated) or′ KingFisher™ system (Thermo Labsystems))

Three target capture oligonucleotides were designed for the P. acnes 16S rRNA, two specific oligonucleotides that correspond to bases 221 and 250 of E. coli accession number V00331 and GI number 42756 (SEQ ID Nos. 127-130) and one non-specific capture oligonucleotide. Use of non-specific capture oligonucleotides has been described previously (see US 2008-0286775 A1, Becker et al.). Briefly, the non-specific capture probes used in the following tests are oligonucleotides of random sequences of G's and U's, 18 bases in length that non-specifically bind to nucleic acid. Target capture oligonucleotides were tested to confirm they did not interfere with the amplification reaction. Specifically, 2.5 pmol of target capture oligomers (SEQ ID Nos. 127 or 129) or 2.5 pmol of non-specific capture probe were spiked in samples containing 10⁵ copies of target RNA. Samples were amplified using real-time single primer transcription associated amplification with one of the assay oligomer combinations described earlier herein. Target capture oligonucleotides exhibited minimal affects on the amplification reaction, data shown in Table 13.

TABLE 13
Affect of Target Capture Oligonucleotides on Amplification

| | AveRange (RFU) | TTime (minutes) |
|---|---|---|
| No Target Capture Oligomer | 1.270 | 15.0 |
| SEQ ID NO. 127 | 1.035 | 14.3 |
| SEQ ID NO. 129 | 1.030 | 14.0 |
| Non-Specific Capture Probe | 0.940 | 14.6 |

Target capture oligomer sensitivity was determined by: (1) keeping the amount of P. acnes 16S rRNA constant and varying the amount of target capture oligomers, and (2) keeping the amount of target capture oligomers constant and varying the amount of P. acnes 16S rRNA. The first test, 0 or 10⁶ copies of P. acnes 16S rRNA were amplified using 0, 2.5, 5, 10, or 20 picomoles of capture probe per mL of lysis reagent. In the second test, 0, 10³, 10⁴, 10⁵, and 106 copies of P. acnes 16S rRNA were amplified using 5 picomoles of capture probe per mL of lysis reagent. In both tests, nucleic acids samples were amplified using real time single primer transcription associated amplification, using assay oligomer combination 1. Data from the sensitivity experiments is shown in Tables 14 and 15.

TABLE 14
Sensitivity of 16S Target Capture Oligonucleotides Test 1

| Target Capture | Amt. Target | AveRange | TTime |
|---|---|---|---|
| Probe | Capture | (RFU) | (minutes) |
| SEQ ID NO. 127 | 0 | 0.178 | 17.3 |
| | 2.5 | 0.330 | 15.0 |
| | 5 | 0.355 | 15.3 |
| | 10 | 0.354 | 15.5 |
| | 20 | 0.351 | 16.4 |
| SEQ ID NO. 129 | 0 | 0.196 | 19.2 |
| | 2.5 | 0.321 | 15.1 |
| | 5 | 0.315 | 15.0 |
| | 10 | 0.335 | 16.0 |
| | 20 | 0.334 | 16.1 |
| Non-specific | 0 | 0.177 | 17.6 |
| Capture Probe | 2.5 | 0.332 | 17.0 |
| | 5 | 0.338 | 16.9 |
| | 10 | 0.306 | 15.3 |
| | 20 | 0.310 | 17.0 |

TABLE 15
Sensitivity of 16S Target Capture Oligonucleotides Test 2

| Target Capture | Amt. P. acnes | AveRange | TTime |
|---|---|---|---|
| Probe | rRNA | (RFU) | (minutes) |
| SEQ ID NO. 127 | 0 | 0.173 | 17.3 |
| | 10³ | 0.194 | 16.7 |
| | 10⁴ | 0.200 | 17.9 |
| | 10⁵ | 0.309 | 16.7 |
| | 10⁶ | 0.356 | 14.3 |
| SEQ ID NO. 129 | 0 | 0.146 | 16.0 |
| | 10³ | 0.169 | 16.2 |
| | 10⁴ | 0.186 | 16.2 |
| | 10⁵ | 0.293 | 16.5 |
| | 10⁶ | 0.333 | 14.2 |
| Non-Specific | 0 | 0.158 | 17.4 |
| Capture Probe | 10³ | 0.140 | 16.0 |
| | 10⁴ | 0.168 | 18.3 |
| | 10⁵ | 0.229 | 17.9 |
| | 10⁶ | 0.301 | 16.3 |

Target capture oligonucleotides were also tested for cross reactivity against the following challenge organisms: P. cyclohexanicum, P. avidum and P. theonii. Briefly, target capture probes were added to samples containing the 10⁴ CFU of challenge organism. Controls were run containing 0 or 10⁶ copies of P. acnes 16S rRNA. All samples were amplified using real time single primer transcription associated amplification, using assay oligomer combination 1. Data is show in Table 16.

TABLE 16
Cross Reactivity of Target Capture Oligomers

| | Amt. | | | |
|---|---|---|---|---|
| Target Capture | P. acnes | Challenge | AveRange | TTime |
| Probe | 16S rRNA | Organism | (RFU) | (Minutes) |
| SEQ ID NO. | 0 | none | 0.123 | 20.8 |
| 127 | 10⁶ | none | 0.461 | 16.1 |
| | 0 | P. avidum | 0.059 | 7.8 |
| | 0 | P. cyclohexanicum | 0.125 | 19.3 |
| | 0 | P. theonii | 0.062 | 7.8 |
| SEQ ID NO. | 0 | none | 0.136 | 19.8 |
| 129 | 10⁶ | none | 0.525 | 16.7 |
| | 0 | P. avidum | 0.049 | 5.7 |
| | 0 | P. cyclohexanicum | 0.137 | 20.8 |
| | 0 | P. theonii | 0.067 | 5.9 |

Target capture oligomers were further tested for cross reactivity to ensure the challenge organisms, P. cyclohexanicum, P. avidum and P. theonii, did not inhibit amplification. Briefly, target capture probes were added to samples containing 0, 10³, 10⁴, 10⁵, or 10⁶ copies of P, acnes 16S rRNA with and without the 10⁴ CFU of the challenge organism. Following target capture, all samples were amplified using real time single primer transcription associated amplification, using assay oligomer combination 1 Results of the tests in which target capture was performed using SEQ ID NO. 129 are shown in Table 17.

TABLE 17
Specific Amplification of P. acnes 16S rRNA in the Presence
of Challenge Organisms

| Challenge | Amt. P. acnes | AveRange | TTime |
|---|---|---|---|
| Organism | rRNA | (RFU) | (minutes) |
| None | 0 | 0.105 | 13.3 |
| | 10³ | 0.109 | 15.7 |
| | 10⁴ | 0.153 | 19.8 |
| | 10⁵ | 0.306 | 23.3 |
| | 10⁶ | 0.430 | 20.4 |
| P. avidum | 0 | 0.067 | 6.5 |
| | 10³ | 0.066 | 9.3 |
| | 10⁴ | 0.074 | 4.2 |
| | 10⁵ | 0.068 | 6.5 |
| | 10⁶ | 0.098 | 9.6 |
| P. cyclohexanicum | 0 | 0.097 | 10.9 |
| | 10³ | 0.105 | 12.1 |
| | 10⁴ | 0.193 | 18.2 |
| | 10⁵ | 0.348 | 24.0 |
| | 10⁶ | 0.580 | 21.2 |
| P. theonii | 0 | 0.073 | 8.4 |
| | 10³ | 0.077 | 6.8 |
| | 10⁴ | 0.080 | 7.6 |
| | 10⁵ | 0.094 | 7.9 |
| | 10⁶ | 0.324 | 19.8 |

### Example 5

### TAG Amplification and Real Time Detection of P. acnes 16S rRNA

Methods for reducing background fluorescence are known and one such method, called TAG amplification, was used to reduce the background signal for real time detection of P. acnes 16S rRNA. TAG amplification has been described in detail previously (see US. 2007-0281317 A1 Becker et al.). Briefly, TAG amplification modifies one of the amplification oligomers (referred to as the TAG oligomer) to include a non-target binding sequence at the 5′ end. TAG oligomers may be included with the target capture probe during the target capture step. Thus, the sample nucleic acid simultaneously hybridizes to both the target capture probe and the TAG oligomer. All non-bound nucleic acid is washed away, including excess TAG oligomers. Samples are amplified, the first round of amplification uses the TAG oligomer whereas subsequent rounds of amplification use oligomers specific for the TAG sequence. Embodiments of TAG oligomers for amplifying P. acnes 16S rRNA are shown in Table 18, in which the lower case letters represent the TAG sequences.

TABLE 18
P. acnes 16S rRNA TAG Oligonucleotides

| Seq | |
|---|---|
| ID | Sequence |
| 135 | GaacctagttgggcgagttacggaCCTGAAGTTATCCCAAAGTCAAG |
| | GGCAGGTT |
| 136 | CCTGAAGTTATCCCAAAGTCAAGGGCAGGTT |
| 137 | AACCTGCCCTgactggtacatgcaagctacCCTGAAGTTATCCCAAA |
| | GTCAAGGGCAGGTT |
| 138 | AACCTGCCCTTGgactggtacatgcaagctacCCTGAAGTTATCCCA |
| | AAGTCAAGGGCAGGTT |
| 139 | AACCTGCCCTTGACgactggtacatgcaagctacCCTGAAGTTATCG |
| | CAAAGTCAAGGGCAGGTT |
| 140 | AACCTGCCCTTGACTTgactggtacatgcaagctacCCTGAAGTTAT |
| | CCCAAAGTCAAGGGCAGGTT |
| 141 | AACCTGCCCTgttgacgtaccgtattgaCCTGAAGTTATCCCAAAGT |
| | CAAGGGCAGGTT |
| 142 | AACCTGCCCTTGgttgacgtaccgtattgaCCTGAAGTTATCCCAAA |
| | GTCAAGGGCAGGTT |
| 143 | AACCTGCCCTTGACgttgacgtaccgtattgaCCTGAAGTTATCCCA |
| | AAGTCAAGGGCAGGTT |
| 144 | AACCTGCCCTTGACTTgttgacgtaccgtattgaCCTGAAGTTATCC |
| | CAAAGTCAAGGGCAGGTT |
| 145 | AACCTGCCCTcattggcatagctagaccCCTGAAGTTATCCCAAAGT |
| | CAAGGGCAGGTT |
| 146 | AACCTGCCCTTGcattggcatagctagaccCCTGAAGTTATCCCAAA |
| | GTCAAGGGCAGGTT |
| 147 | AACCTGCCCTTGACcattggcatagctagaccCCTGAAGTTATCCCA |
| | AAGTCAAGGGCAGGTT |
| 148 | AACCTGCCCTTGACTTcattggcatagctagaccCCTGAAGTTATCC |
| | CAAAGTCAAGGGCAGGTT |
| 149 | AACCTGCCCTTGACTTTgtgacccaatgatctaacaCCTGAAGTTAT |
| | CCCAAAGTCAAGGGCAGGTT |
| 150 | AACCTGCCCTTGACgtgacccaatgatctaacaCCTGAAGTTATCCC |
| | AAAGTCAAGGGCAGGTT |
| 151 | AACCTGCCCTTGgtgacccaatgatctaacaCCTGAAGTTATCCCAA |
| | AGTCAAGGGCAGGTT |
| 152 | AACCTGCCCTgtgacccaatgatctaacaCCTGAAGTTATCCCAAAG |
| | TCAAGGGCAGGTT |
| 153 | AACCTGCCgtgacccaatgatctaacaCCTGAAGTTATCCCAAAGTC |
| | AAGGGCAGGTT |
| 154 | gtgacccaatgatctaacaCCTGAAGTTATCCCAAAGTCAAGGGCAG |
| | GTT |
| 155 | gtgacccaatgatctaacaGAGCACCCCACAAAAGCAG |
| 156 | CTGCTTTTGTGGGGTGCTCTGTgtgacccaatgatctaacaGAGCAC |
| | CCCACAAAAGCAG |
| 157 | CTGCTTTTGTGGGGTGCTCgtgacccaatgatctaacaGAGCACCCC |
| | ACAAAAGCAG |
| 158 | CTGCTTTTGTgtgacccaatgatctaacaGAGCACCCCACAAAAGCA |
| | G |
| 159 | CTGCTTTTgtgacccaatgatctaacaGAGCACCCCACAAAAGCAG |
| 160 | CTGCTTgtgacccaatgatctaacaGAGCACCCCACAAAAGCAG |
| 189 | Gaacctagttgggcgagttacgga |
| 190 | Gactggtacatgcaagctac |
| 191 | Gttgacgtaccgtattga |
| 192 | Cattggcatagctagacc |
| 193 | Gtgacccaatgatctaaca |

Initial testing using TAG amplification was conducted on samples containing 0 or 10⁵ copies of P. acnes 16S rRNA. Target capture was performed using a target specific capture probe and the TAG oligomer. Real time single primer transcription associated amplification was performed, using a variation of assay oligomer combination 1. The variation included: (1) substituting SEQ ID NO. 135 for SEQ ID NO. 20 and (2) adding an additional amplification oligomer SEQ ID NO. 189. TAG amplification greatly reduced the background fluorescence, however, the emergence time was delayed and there were several false positives. Several experiments were conducted to eliminate the false positives by varying the amplification conditions. Zero or 10⁵ copies of P. acnes were amplified with and without blocker oligomers added during target capture, with and without extra blocker oligomers added during amplification, and with or without an additional incubation at 60 or 65° C. for five minutes, after target capture. Due to the ubiquitous nature of P. acnes, the false positives may be due to contamination, rather than problems with the amplification system. Data is shown in Table 19.

TABLE 19
Initial Testing Using the TAG System to Amplify P. acnes 16S rRNA

| | Amt. P. acnes | AveRange | TTime | False | False |
|---|---|---|---|---|---|
| Amplification System | rRNA | (RFU) | (minutes) | Positives | Negatives |
| Single Primer Transcription | 0 | 0.258 | 20.7 | 0 | 0 |
| Associated Amplification | 10⁵ | 0.554 | 18.0 | | |
| TAG | 0 | 0.433 | 34.1 | 3/5 | 0 |
| | 10⁵ | 0.607 | 29.5 | | |
| TAG more replicates | 0 | 0.114 | 33.1 | 2/12 | 0 |
| | 10⁵ | 0.493 | 28.7 | | |
| TAG plus extra incubation | 0 | 0.173 | 35.5 | 1/5 | 1/5 |
| | 10⁵ | 0.725 | 33.0 | | |
| TAG plus extra incubation | 0 | 0.044 | 5.7 | 0/5 | 0/5 |
| and extra blocker | 10⁵ | 0.721 | 32.0 | | |

Modified TAG oligomers were designed (SEQ ID Nos. 137-160) to reduce the false positives and false negatives. The modified TAG oligomers form a hairpin structure using a closing sequence that hybridizes to all or part of the target binding sequence and or the TAG sequence. The closing sequence ranges from 6-22 bases in length. The target binding region of a hairpin TAG oligomer will preferentially bind to the target, thus unfolding the hairpin, rather than stay in the hairpin conformation. The hairpin reduces non-target hybridization of the TAG oligomer. Samples were tested using assay oligomer combination 2 with two changes. SEQ ID NO. 20 was substituted with a sequence selected from SEQ ID Nos. 137-160. If SEQ ID Nos. 137-140 were used, then SEQ ID NO. 190 was added to the amplification oligomers. If SEQ ID Nos. 141-144 were used, then SEQ ID NO. 191 was added to the amplification oligomers. If SEQ ID Nos. 145-148 were used, then SEQ ID NO. 192 was added to the amplification oligomers. If SEQ ID Nos. 149-160 were used, then SEQ ID NO. 193 was added to the amplification oligomers. Samples were amplified using real time single primer transcription associated amplification. Data is shown in Table 20 for SEQ ID Nos. 137-148.

TABLE 20
Initial Testing of the TAG oligomer redesign for P. acnes 16S rRNA

| SEQ ID NO. 20 | Copies P. acnes | AveRange | |
|---|---|---|---|
| Substitution | rRNA | (RFU) | TTime (Minutes) |
| SEQ ID NO. 137 | 0 | 0.055 | No signal |
| | 10⁵ | 0.913 | 24.1 |
| SEQ ID NO. 138 | 0 | 0.058 | No signal |
| | 10⁵ | 0.711 | 25.2 |
| SEQ ID NO. 139 | 0 | 0.052 | No signal |
| | 10⁵ | 0.745 | 26.5 |
| SEQ ID NO. 140 | 0 | 0.062 | No signal |
| | 10⁵ | 0.745 | 25.5 |
| SEQ ID NO. 141 | 0 | 0.503 | 36.2 |
| | 10⁵ | 0.424 | 25.6 |
| SEQ ID NO. 142 | 0 | 0.075 | No signal |
| | 10⁵ | 0.350 | 26.5 |
| SEQ ID NO. 143 | 0 | 0.217 | 35.4 |
| | 10⁵ | 0.415 | 26.4 |
| SEQ ID NO. 144 | 0 | 0.366 | 35.5 |
| | 10⁵ | 0.383 | 26.7 |
| SEQ ID NO. 145 | 0 | 0.964 | 25.6 |
| | 10⁵ | 0.912 | 17.2 |
| SEQ ID NO. 146 | 0 | 0.716 | 25.9 |
| | 10⁵ | 0.760 | 17.7 |
| SEQ ID NO. 147 | 0 | 0.679 | 25.7 |
| | 10⁵ | 0.982 | 19.0 |
| SEQ ID NO. 148 | 0 | 0.336 | 25.5 |
| | 10⁵ | 0.800 | 21.1 |

SEQ ID Nos. 137-140 provided the best background signal reduction and were further tested for sensitivity. The oligomer combinations were tested at 0, 10⁴ and 10⁵ copies of P. acnes 16S rRNA, the data is shown in Table 21. Assay oligomer combination with SEQ ID NO. 137 was further tested for sensitivity. The SEQ ID NO. 137 oligomer combination resulted in 1/15 false positives and no false negatives at 10⁴ and 10⁵ copies of 16S rRNA, and 1/15 false negatives at 10³ copies.

TABLE 21
Sensitivity Testing for the TAG Oligomer Redesigned for P. acnes
16s rRNA

| SEQ ID NO. 20 | Copies P. acnes | AveRange | TTime | False |
|---|---|---|---|---|
| Substitution | rRNA | (RFU) | (Minutes) | negatives |
| SEQ ID NO. 137 | 0 | 0.063 | 31.4 | 1/6 |
| | 10⁴ | 0.790 | 30.1 | |
| | 10⁵ | 1.013 | 26.3 | |
| SEQ ID NO. 138 | 0 | 0.104 | 33.0 | 3/6 |
| | 10⁴ | 0.526 | 29.6 | |
| | 10⁵ | 0.993 | 27.4 | |
| SEQ ID NO. 139 | 0 | 0.039 | No signal | 5/6 |
| | 10⁴ | 0.204 | 33.9 | |
| | 10⁵ | 1.062 | 27.8 | |
| SEQ ID NO. 140 | 0 | 0.043 | No signal | 3/6 |
| | 10⁴ | 0.300 | 30.6 | |
| | 10⁵ | 0.887 | 29.8 | |

### Example 6

### Increasing Sensitivity in P. acnes 16S rRNA Detection

Experiments were conducted to determine if sensitivity may be improved by adding one or more helper probes, selected from SEQ ID Nos. 195-198 (see Table 22), to the target capture step. Helper probes have been described previously (see U.S. Pat. No. 5,030,557, Hogan). Briefly, helper probes are short probes that help reduce hybridization inhibition due to secondary structure. Their small size allows them to bind areas near secondary structures and then the secondary structure changes which allows a longer oligomer to hybridize to the nucleic acid.

TABLE 22
Target Capture Helper Oligomers for P. acnes 16S rRNA

| SEQ ID NO. | Sequence |
|---|---|
| 195 | CATGCAGCAGGAGCTCCTATC |
| 196 | GCTCCTATCCGGTATTAGC |
| 197 | GCAGGAGCTCCTATCCGGTA |
| 198 | GCAGGAGCTCCTATCCGGTATTAGC |

Samples were tested using assay oligomer combination 2 with two changes: SEQ ID NO. 141 replaced SEQ ID NO. 20, and SEQ ID NO. 191 was added to the amplification oligonucleotides. The first test compared samples with and without helper probes, the second test compared the different helper probes. Both tests used real time single primer transcription associated amplification. In the first test, 0, 10³ or 10⁵ copies of P. acnes 16S rRNA underwent target capture using a specific capture probe (SEQ ID NO. 129) with and without helper probe (SEQ ID NO. 195). The results of the first test did not improve sensitivity, but did improve the slope of the curve. In the second test, 0 or 10⁴ copies of P. acnes 16S rRNA underwent target capture using a specific capture probe (SEQ ID NO. 129) and one helper probe chosen from SEQ ID Nos. 196-198. The results of the second test showed amplification inhibition or problems with the experimental set up because data for SEQ ID NO. 195 is lower than previous tests. Data for the two tests is show in Tables 23 and 24, “AveSlope” is the average slope of the real time amplification curve; “AveRange” stands for average (mean) of detected RFU for replicate samples tested in the same condition.

TABLE 23
P. acnes Amplified With and Without Target Capture Helper Probes

| | Copies P. acnes | | AveRange | TTime |
|---|---|---|---|---|
| | rRNA | AveSlope | (RFU) | (minutes) |
| No helper | 0 | 0.018 | 0.040 | No signal |
| | 10³ | 0.026 | 0.099 | 29.0 |
| | 10⁵ | 0.050 | 0.603 | 26.0 |
| With helper | 0 | 0.024 | 0.092 | 28.3 |
| SEQ ID NO. 195 | 10³ | 0.025 | 0.098 | 27.1 |
| | 10⁵ | 0.063 | 0.716 | 25.2 |

TABLE 24
P. acnes Comparison of Target Capture Helper Probes

| | Copies P. acnes | | AveRange | TTime |
|---|---|---|---|---|
| Helper Probe | rRNA | AveSlope | (RFU) | (minutes) |
| SEQ ID NO. 195 | 0 | 0.019 | 0.083 | 20.7 |
| | 10⁴ | 0.018 | 0.100 | 14.1 |
| SEQ ID NO. 196 | 0 | 0.018 | 0.063 | No signal |
| | 10⁴ | 0.019 | 0.075 | No signal |
| SEQ ID NO. 197 | 0 | 0.020 | 0.062 | No signal |
| | 10⁴ | 0.018 | 0.073 | 9.1 |
| SEQ ID NO. 198 | 0 | 0.019 | 0.064 | No signal |
| | 10⁴ | 0.018 | 0.086 | 20.5 |

### Example 7

### Reducing False Positives with Base Treatment

Due to the prevalence of P. acnes in the environment and on human skin, buffers and oligomers that are made in the presence of people may contain trace amounts of P. acnes thus causing false positives. Pre-treating amplification oligomers and buffers with base may reduce false positives by degrading any P. acnes RNA that is present in the reagents. One method for base treating the oligomers uses lysis reagent (see Example 1) that is not brought to pH 7.5; this will be referred to as base or basic lysis reagent. Target capture and some of the amplification oligomers are added to the base lysis reagent, heated at 60° C. for 90 minutes and cooled to room temperature. After cooling, the base lysis reagent is neutralized by injecting hydrochloric acid into the tube using a sterile syringe. Once neutralized, the sample may be added to the lysis buffer and oligomers.

### Example 8

### Reducing False Positives with Modified Target Capture Probes

One method to reduce false positives uses target capture probes that have been modified to include a hairpin formation. Hairpin target capture probes have been described previously (see US 2006-0068417 A1 Becker et al). Embodiments of hairpin target capture probes for capturing P. acnes nucleic acid are listed in Table 25, in which lower case letters represent the hairpin closing sequence.

TABLE 25
Hairpin Target Capture Oligomers

| SEQ ID | |
|---|---|
| NO | Sequence |
| 219 | GCUGAUAAGCCGCGAGUTTTAAAAAAAAAAAAAAAAAAAAAAAA |
| | AAAAAA |
| 220 | GCUGAUAAGCCGCGAGUaucagcTTTAAAAAAAAAAAAAAAAAA |
| | AAAAAAAAAAAA |
| 221 | GCUGAUAAGCCGCGAGUuaucagcTTTAAAAAAAAAAAAAAAAA |
| | AAAAAAAAAAAAA |
| 222 | GCUGAUAAGCCGCGAGUuuaucagcTTTAAAAAAAAAAAAAAAA |
| | AAAAAAAAAAAAAA |
| 223 | GCUGAUAAGCCGCGAGUcuuaucagcTTTAAAAAAAAAAAAAAA |
| | AAAAAAAAAAAAAAA |
| 224 | GCUGAUAAGCCGCGAGUgcuuaucagcTTTAAAAAAAAAAAAAA |
| | AAAAAAAAAAAAAAAA |
| 225 | GCUGAUAAGCCGCGAGUcggcuuaucagcTTTAAAAAAAAAAAA |
| | AAAAAAAAAAAAAAAAAA |
| 226 | GCUGAUAAGCCGCGAGUcgcggcuuaucagcTTTAAAAAAAAAA |
| | AAAAAAAAAAAAAAAAAAAA |

Linear target capture probes (SEQ ID NO. 219) were compared to hairpin target capture probes with hairpin closing sequences that were 10, 12, or 14 bases long (SEQ ID Nos. 224, 225, and 226 respectively). Target capture was preformed with the target capture probes and amplification oligomers (SEQ ID Nos. 210 and 8). After separating the target capture complexes, the samples underwent real-time single primer transcription associated amplification using SEQ ID Nos. 35, 193 and 181. Results are shown in Table 26. For the linear target capture probes, 6 out of 12 negative samples were positive. For the hairpin target capture probes with 10 or 12 base closing sequences, 1 out of 12 negative samples were positive. For the hairpin target capture probes with 14 base closing sequence there were no false positives. Although the hairpin target capture probes reduced the number of false positives, they also reduced the sensitivity of the assay.

TABLE 26
Linear and Hairpin Target Capture Probes

| Target Capture | Copies | AveRange | TTime |
|---|---|---|---|
| Probe | rRNA | (RFU) | (minutes) |
| SEQ ID NO. 219 | 0 | 0.202 | 28.6 |
| | 10⁴ | 0.471 | 23.3 |
| | 10⁵ | 0.505 | 22.5 |
| SEQ ID NO. 224 | 0 | 0.023 | 28.0 |
| | 10⁴ | 0.024 | 27.5 |
| | 10⁵ | 0.353 | 28.2 |
| SEQ ID NO. 225 | 0 | 0.026 | 27.2 |
| | 10⁴ | 0.209 | 29.5 |
| | 10⁵ | 0.405 | 28.9 |
| SEQ ID NO. 226 | 0 | 0.026 | 0 |
| | 10⁴ | 0.207 | 28.0 |
| | 10⁵ | 0.143 | 29.9 |

To improve sensitivity while reducing false positives, hairpin target capture was combined with base treating the target capture probes and some of the amplification oligomers as described in Example 7. The closing sequences for the hairpin target capture oligomer were reduced to 7, 8, or 9 bases in length (SEQ ID Nos. 221, 222, or 223 respectively). SEQ ID Nos. 210 and 8 were added to a tube containing base lysis buffer with either SEQ ID Nos. 219 or 221 or 222 or 223 and heated at 60° C. for 90 minutes. The tubes were cooled to room temperature and were sterilely injected with hydrochloric acid to neutralize the buffer. After separating the target capture complexes, the samples underwent real-time single primer transcription associated amplification using SEQ ID Nos. 35, 193 and 181. The results are shown in Table 27. The base treatment produced no false positives, even when using the linear target capture oligomer. Additionally, the assay sensitivity was equivalent between the linear target capture probe and the hairpin target capture probe.

TABLE 27
Linear and Hairpin Target Capture Probes with Base Treatment

| Target Capture | Copies | AveRange | TTime |
|---|---|---|---|
| Probe | rRNA | (RFU) | (minutes) |
| SEQ ID NO. 219 | 0 | 0.041 | 0 |
| | 10⁴ | 0.415 | 27.1 |
| | 10⁵ | 0.413 | 24.0 |
| SEQ ID NO. 221 | 0 | 0.031 | 0 |
| | 10⁴ | 0.415 | 27.4 |
| | 10⁵ | 0.408 | 24.0 |
| SEQ ID NO. 222 | 0 | 0.044 | 23.0 |
| | 10⁴ | 0.604 | 26.6 |
| | 10⁵ | 0.613 | 23.8 |
| SEQ ID NO. 223 | 0 | 0.047 | 12.8 |
| | 10⁴ | 0.578 | 24.5 |
| | 10⁵ | 0.596 | 22.5 |

### Example 9

### Reducing False Positives with Scavenger Oligomers

Scavenger oligomers are short oligomers (generally less than 20 bases) that are designed to hybridize to non-target bound oligomers, e.g. excess amplification oligomers or capture probes. Hybridizing the excess oligomers with scavengers helps prevent the excess oligomers from non-specifically hybridizing to non-target material. Scavenger oligomers are generally designed to hybridize to a region on either the target capture oligomer or the amplification oligomer that may potentially hybridize to a non-specific target. For example, target capture probes have a poly A tail, therefore any poly-T sites in a non-specific target may non-specifically hybridize to the poly-A tail. Alternatively, scavengers may hybridize to a structural region such as a hairpin. Examples of scavenger oligomers are listed in Table 28 below. SEQ ID Nos. 211, 212, 213, 214, 215, and 216 may be used with a TAG oligomer (SEQ ID NO. 210). SEQ ID Nos. 217 and 218 may be used with a target capture oligomer of SEQ ID Nos. 219, 220, 221, 222, 223, 224, 225, or 226 (see Table 25 above).

TABLE 28
Scavenger Oligonucleotides

| | |
|---|---|
| SEQ ID NO | Sequence |

| | |
|---|---|
| 210 | CTGCTTTTGTGGGGgtgacccaatgatctaacaGAGCACCC |
| | CACAAAAGCAG |
| 211 | CTGCTTTTGTGGGGTG |
| 212 | CTGCTTTTGTGGGG |
| 213 | CTGCTTTTGTGG |
| 214 | CTGCTTTTGT |
| 215 | GTGGGGTGCTCTGT |
| 216 | GGGGTGCTCTGTTAG |
| 217 | UUUUUUUAAAACUCGCGGCU |
| 218 | TTTTTTTAAAACTCGCGGCT |

Scavengers oligomers were added to either the target capture probes or the amplification oligomers. For assays where scavenger oligomers were added to the target probes, one scavenger oligomer was selected from SEQ ID Nos. 211-218 and added to SEQ ID NO. 219, 210, and 8. Zero or 10⁴ copies of P. acnes rRNA was added and target capture was performed as described in Example 4. After target capture the samples were amplified by real-time single primer transcription associated amplification, as described in Example 3 using SEQ ID Nos. 8, 35, 181, and 193. For assays where scavenger oligomers were added to the amplification oligomers, target capture was performed using SEQ ID Nos. 219, 210, and 8 on zero or 10⁴ copies of P. acnes rRNA. After target capture the samples were amplified by real-time single primer transcription associated amplification using SEQ ID Nos. 8, 35, 181, and 193, plus one sequence selected from SEQ ID Nos. 211-218. Results are shown in Tables 28 and 30. Adding scavenger oligomers to the target capture probes did not eliminate false positives (results reported below are number of false positives per 15 total assays performed for each condition reported). Adding scavenger oligomers to the amplification oligomers did not inhibit amplification and SEQ ID Nos. 215 and 217 did not have any false positives.

TABLE 29
Scavenger Oligomers Added to Target Capture

| Scavenger | Copies | AveRange | TTime | False Positives |
|---|---|---|---|---|
| Oligomer | rRNA | (RFU) | (minutes) | per 15 assays |
| No scavenger | 0 | 1852.47 | 20.77 | 3 |
| oligomer | 10⁴ | 10314.00 | 15.43 | |
| SEQ ID NO. 211 | 0 | 1606.53 | 24.33 | 3 |
| | 10⁴ | 10588.78 | 15.73 | |
| SEQ ID NO. 212 | 0 | 1377.40 | 21.69 | 2 |
| | 10⁴ | 10816.67 | 15.59 | |
| SEQ ID NO. 213 | 0 | 1486.00 | 28.72 | 4 |
| | 10⁴ | 10827.00 | 15.35 | |
| SEQ ID NO. 214 | 0 | 3650.87 | 20.03 | 5 |
| | 10⁴ | 11179.89 | 16.17 | |
| SEQ ID NO. 215 | 0 | 768.93 | 16.84 | 2 |
| | 10⁴ | 10537.89 | 16.38 | |
| SEQ ID NO. 216 | 0 | 1573.60 | 24.35 | 2 |
| | 10⁴ | 10911.11 | 16.69 | |
| SEQ ID NO. 217 | 0 | 4026.00 | 20.25 | 8 |
| | 10⁴ | 11943.33 | 14.12 | |
| SEQ ID NO. 218 | 0 | 3997.93 | 16.10 | 6 |
| | 10⁴ | 11163.33 | 14.45 | |

TABLE 30
Scavenger Oligomers Added to Amplification Oligomers

| Scavenger | Copies | AveRange | TTime | False Positives |
|---|---|---|---|---|
| Oligomer | rRNA | (RFU) | (minutes) | per 15 assays |
| No scavenger | 0 | 2114.67 | 24.52 | 4 |
| oligomer | 10⁴ | 11133.89 | 15.94 | |
| SEQ ID NO. 211 | 0 | 1183.33 | 20.41 | 1 |
| | 10⁴ | 9467.11 | 15.75 | |
| SEQ ID NO. 212 | 0 | 2268.33 | 25.67 | 3 |
| | 10⁴ | 10056.00 | 15.64 | |
| SEQ ID NO. 213 | 0 | 1276.93 | 20.33 | 3 |
| | 10⁴ | 10000.56 | 15.80 | |
| SEQ ID NO. 214 | 0 | 1937.13 | 21.51 | 3 |
| | 10⁴ | 10274.67 | 15.74 | |
| SEQ ID NO. 215 | 0 | 657.47 | n/a | |
| | 10⁴ | 10306.44 | 15.41 | |
| SEQ ID NO. 216 | 0 | 1854.33 | 22.69 | 5 |
| | 10⁴ | 10293.33 | 16.24 | |
| SEQ ID NO. 217 | 0 | 683.47 | n/a | |
| | 10⁴ | 10018.44 | 15.11 | |
| SEQ ID NO. 218 | 0 | 613.47 | 25.59 | 1 |
| | 10⁴ | 9820.78 | 15.99 | |

### Example 10

### Design and Initial Testing of P. acnes 23S Amplification Oligomer Sets

Amplification and detection oligonucleotides were designed to amplify and detect P. acnes 23S rRNA. In general, amplification and detection were targeted to bases 608,745-609,295 of accession number AE017283 and GI number 50839098 (SEQ ID NO. 202). The first set of amplification and detection oligomers corresponds to bases 608,775-609,045 (SEQ ID NO. 203) and the second set of amplification and detection oligomers corresponds to bases 608,995-609,275 (SEQ ID NO. 204) of accession number AEO 17283 and GI number 50839098 (SEQ ID Nos. 9-15, 27-34, 55-72, 99-126,183, and 185-188). Known amounts of P. acnes 23S rRNA sequences were amplified using real time transcription associated amplification using combinations of the oligomers in Tables 4 and 5.

Amplification oligonucleotide combinations were tested at 0 and 105 copies of P. acnes 23S rRNA, under conditions similar to Example 3. Oligomer combinations were evaluated on signal emergence time, maximum RFU signal, slope of the sigmoid curve, and background signal levels. Assay oligomer combinations targeting the first region of P. acnes 23S rRNA were: (1) SEQ ID Nos. 11, 27, 61 and 107; (2) SEQ ID Nos. 11, 32, 61, and 105; (3) SEQ ID Nos. 11, 27, 61, and 99; (4) SEQ ID Nos. 11, 32, 61, and 103; (5) SEQ ID Nos. 11, 31, 61, and 103; and (6) SEQ ID Nos. 11, 32, 59, and 101. Assay oligomer combinations, targeting the second region of P. acnes 23S rRNA, were: (7) SEQ ID Nos. 14, 34, 69, and 117; (8) SEQ ID Nos. 14, 34, 67, and 117; (9) SEQ ID Nos. 14, 33, 67, and 117; (10) SEQ ID Nos. 14, 33, 67, and 119; and (11) SEQ IDNos. 183, 185, 186, and 188. Data for assay oligomer combinations 2 and 11 are shown in Table 31.

TABLE 31
Data for oligomer combinations 2 and 11 amplifying 23S rRNA

| Amplification | Copies | AveRange | TTime |
|---|---|---|---|
| Oligonucleotides | rRNA | (RFU) | (minutes) |
| Combination 2 | 0 | 0.107 | 25.3 |
| | 10³ | 0.110 | 27.8 |
| | 10⁴ | 0.921 | 20.8 |
| | 10⁵ | 2.145 | 16.4 |
| Combination 11 | 0 | 3.191 | 44.4 |
| | 10³ | 2.333 | 34.7 |
| | 10⁴ | 2.556 | 30.5 |
| | 10⁵ | 2.618 | 26.6 |

### Example 11

### TAG Amplification Oligomers for P. acnes 23S rRNA

Oligomers that may be used to amplify P. acnes 23S rRNA using the TAG amplification system are listed in Table 32, lowercase letters represent the TAG sequence. As described in Example 5, this method is used to reduce background fluorescence in a real time detection assay. Using selected combinations of assay oligomers from the group shown in Table 32, amplification is performed substantially as described in Example 5, but using 23S RNA transcripts for the target for amplification. The amplified product is detected during amplification (i.e., in real time) by using a probe, such as a molecular beacon or molecular torch, that hybridizes specifically to the amplified product made from the amplified tag sequence or specifically hybridizes to the amplified 23S sequence located between the selected amplification oligomers in Table 32, which include those selected from the 23S rRNA sequence specific probes are shown in Table 6.

TABLE 32
23S rRNA TAG Amplification Oligomers

| SEQ ID | |
|---|---|
| NO | Sequence |
| 161 | GTGTGACGGGTGGGAAgttgacgtaccgtattgaATTCCCACCC |
| | GTCACAC |
| 162 | GTGTGACGGGTGGGgttgacgtaccgtattgaATTCCCACCCGT |
| | CACAC |
| 163 | GTGTGACGGGTGgttgacgtaccgtattgaATTCCCACCCGTCA |
| | CAC |
| 164 | GTGTGACGGGgttgacgtaccgtattgaATTCCCACCCGTCACA |
| | C |
| 165 | GTGTGACGGGTGGGAAcattggcatagctagaccATTCCCACCC |
| | GTCACAC |
| 166 | GTGTGACGGGTGGGcattggcatagctagaccATTCCCACCCGT |
| | CACAC |
| 167 | GTGTGACGGGTGcattggcatagctagaccATTCCCACCCGTCA |
| | CAC |
| 168 | GTGTGACGGGcattggcatagctagaccATTCCCACCCGTCACA |
| | C |
| 169 | GTGTGACGGGTGGGAAgcatgcaggttaacgtagacATTCCCAC |
| | CCGTCACAC |
| 170 | GTGTGACGGGTGGGgcatgcaggttaacgtagacATTCCCACCC |
| | GTCACAC |
| 171 | GTGTGACGGGTGgcatgcaggttaacgtagacATTCCCACCCGT |
| | CACAC |
| 172 | GTGTGACGGGgcatgcaggttaacgtagacATTCCCACCCGTCA |
| | CAC |
| 173 | GTGTGACGGGTGGGAAgtgacccaatgatctaacaATTCCCACC |
| | CGTCACAC |
| 174 | GTGTGACGGGTGGGgtgacccaatgatctaacaATTCCCACCCG |
| | TCACAC |
| 175 | GTGTGACGGGTGgtgacccaatgatctaacaATTCCCACCCGTC |
| | ACAC |
| 176 | GTGTGACGGGgtgacccaatgatctaacaATTCCCACCCGTCAC |
| | AC |
| 177 | TCGTGTCAGTGGTGAAgtgacccaatgatctaacaGCTTCACCA |
| | CTGACACGA |
| 178 | TCGTGTCAGTGGTGgtgacccaatgatctaacaGCTTCACCACT |
| | GACACGA |
| 179 | TCGTGTCAGTGGgtgacccaatgatotaacaGCTTCACCACTGA |
| | CACGA |
| 180 | gtgacccaatgatctaacaGCTTCACCACTGACACGA |
| 191 | gttgacgtaccgtattga |
| 192 | cattggcatagctagacc |
| 193 | gtgacccaatgatctaaca |
| 194 | gcatgcaggttaacgtagac |

1. A composition comprising at least two oligomers specific for a P. acnes 16S rRNA sequence or DNA encoding 16S rRNA contained within SEQ ID NO: 199 selected from the group consisting of SEQ ID Nos. 16 to 26, 35 to 54, 135 to 160, 184, and 210, the completely complementary sequences of SEQ ID Nos. 16 to 26, 35 to 54, 135 to 160, 184, and 210, and the RNA equivalent of SEQ ID Nos. 16 to 26, 35 to 54, 135 to 160, 184, and 210, wherein when a first oligomer of the at least two oligomers is selected from the group consisting of SEQ ID Nos. 16 to 26, 135 to 160, 184 and 210, then a second oligomer is selected from the group consisting of SEQ ID Nos. 35 to 54.

2. The composition of claim 1, further comprising at least one third oligomer selected from the group consisting of SEQ ID Nos. 1 to 8, 75 to 98, 127 to 130, 181, 182, 195 to 198, and 211 to 226, the completely complementary sequences of SEQ ID Nos. 1 to 8, 75 to 98, 127 to 130, 181, 182, 195 to 198, and 211 to 226, and the RNA equivalents of SEQ ID Nos. 1 to 8, 75 to 98, 127 to 130, 181, 182, 195 to 198, and 211 to 226.

3. The composition of claim 1, wherein the at least two oligomers include a promoter provider oligomer selected from the group consisting of SEQ ID Nos. 35 to 54 and 135 to 160.

4. The composition of claim 2, wherein the at least one third oligomer is selected from the group consisting of SEQ ID Nos. 1 to 8, the completely complementary sequences of SEQ ID Nos. 1 to 8, and the RNA equivalent of SEQ ID Nos. 1 to 8, and wherein the third oligomer selected from this group includes a 3′ blocked end.

5. The composition of claim 2 wherein the at least one third oligomer is selected from the group consisting of SEQ ID Nos. 75 to 98, 181, and 182, the completely complementary sequences of SEQ ID Nos. 75 to 98, 181, and 182, and the RNA equivalent of SEQ ID Nos. 75 to 98, 181, and 182, and wherein the third oligomer selected from this group includes a label attached to the third oligomer.

6. A composition comprising at least two oligomers specific for a P. acnes 23S rRNA sequence or DNA encoding 23S rRNA contained within SEQ ID NO:202 selected from the group consisting of SEQ ID Nos. 27 to 34, 55 to 72, 161 to 180, and 185 to 187, the completely complementary sequences of SEQ ID Nos. 27 to 34, 55 to 72, 161 to 180, and 185 to 187, and the RNA equivalent of SEQ ID Nos. 27 to 34, 55 to 72, 161 to 180, and 185 to 187, wherein when a first oligomer of the at least two oligomers is selected from the group consisting of SEQ ID Nos. 27 to 34, 161 to 180, and 185, then a second oligomer is selected from the group consisting of SEQ ID Nos. 55 to 72, 186, and 187.

7. The composition of claim 6, further comprising at least one third oligomer selected from the group consisting of SEQ ID Nos. 9 to 15, 99 to 126, 131 to 134, 183, and 188, the completely complementary sequences of SEQ ID Nos. 9 to 15, 99 to 126, 131 to 134, 183, and 188, and the RNA equivalent of SEQ ID Nos. 9 to 15, 99 to 126, 131 to 134, 183, and 188.

8. The composition of claim 6, wherein the at least two oligomers include a promoter provider oligomer selected from the group consisting of SEQ ID Nos. 55 to 72, 186, and 187, the completely complementary sequences of SEQ ID Nos. 55 to 72, 186, and 187, and the RNA equivalent of SEQ ID Nos. 55 to 72, 186, and 187.

9. The composition of claim 7, wherein the at least one third oligomer is selected from the group consisting of SEQ ID Nos. 9 to 15 and 188, the completely complementary sequences of SEQ ID Nos. 9 to 15 and 188, and the RNA equivalent of SEQ ID Nos. 9 to 15 and 188, and wherein the third oligomer selected from this group includes a 3′ blocked end.

10. The composition of claim 7 wherein the at least one third oligomer is selected from the group consisting of SEQ ID Nos. 99 to 126 and 183, the completely complementary sequences of SEQ ID Nos. 99 to 126 and 183, and the RNA equivalent of SEQ ID Nos. 99 to 126 and 183, and wherein the third oligomer selected from this group includes a label attached to the third oligomer.

11. A method for specifically detecting a P. acnes in a sample by detecting a target nucleic acid sequence that is a P. acnes rRNA or DNA encoding a P. acnes rRNA comprising the steps of:
(a) providing a sample that contains a P. acnes target region in a 16S rRNA or DNA encoding 16S rRNA contained in SEQ ID NO:199, and/or a target region in a 23S rRNA or gene encoding 23S RNA contained in SEQ ID NO:202;
(b) amplifying a sequence in the P. acnes target region to make an amplified product by using at least two amplification oligomers in an in vitro amplification reaction that includes enzymatic elongation of at least one amplification oligomer,
wherein when the target sequence is in the 16S rRNA or DNA encoding the 16S rRNA contained in SEQ ID NO: 199, the at least two amplification oligomers are selected from the group consisting of SEQ ID Nos. 16 to 26, 35 to 54, 135 to 160, 184, and 210, the completely complementary sequences of SEQ ID Nos. 16 to 26, 35 to 54, 135 to 160, 184, and 210, and the RNA equivalent of SEQ ID Nos. 16 to 26, to 54, 135 to 160, 184, and 210, and
wherein when the target sequence is in the 23S rRNA or DNA encoding 23S rRNA contained in SEQ ID NO:202, the at least two amplification oligomers are selected from the group consisting of SEQ ID Nos. 27 to 34, 55 to 72, 161 to 180, and 185 to 187, the completely complementary sequences of SEQ ID Nos. 27 to 34, 55 to 72, 161 to 180, and 185 to 187, and the RNA equivalent of SEQ ID Nos. 27 to 34, 55 to 72, 161 to 180, and 185 to 187; and
(c) detecting the amplified product.

12. The method of claim 11, wherein the target sequence is in the 16S rRNA or DNA encoding the 16S rRNA, the at least two amplification oligomers use a first amplification oligomer selected from the group consisting of SEQ ID Nos. 16 to 26, 135 to 160, 184 and 210, with a second amplification oligomer selected from the group consisting of SEQ ID Nos. 35 to 54.

13. The method of claim 11, wherein the amplifying step for the target sequence in the 16S rRNA or DNA encoding the 16S rRNA further includes a third oligomer selected from the group consisting of SEQ ID Nos. 1 to 8, the completely complementary sequences of SEQ ID Nos. 1 to 8, and the RNA equivalent of SEQ ID Nos. 1 to 8, and wherein the third oligomer selected from this group includes a 3′ blocked end.

14. The method of claim 11, wherein the detecting step for the amplified product made from the target sequence in the 16S rRNA or DNA encoding the 16S rRNA uses a detection probe selected from the group consisting of SEQ ID Nos. 75 to 98, 181, and 182, the completely complementary sequences of SEQ ID Nos. 75 to 98, 181, and 182, and the RNA equivalent of SEQ ID Nos. 75 to 98, 181, and 182.

15. The method of claim 11 further comprising a target capture step to purify the 16S rRNA or DNA encoding the 16S rRNA from the sample by hybridizing to the 16S rRNA or DNA encoding the 16S rRNA a target capture probe selected from the group consisting of SEQ ID Nos. 127 to 130.

16. The method of claim 11, wherein the target sequence is in the 23S rRNA or DNA encoding the 23S rRNA, the at least two amplification oligomers use a first amplification oligomer selected from the group consisting of SEQ ID Nos. 27 to 34, 161 to 180, and 185, with a second amplification oligomer selected from the group consisting of SEQ ID Nos. 55 to 72, 186, and 187.

17. The method of claim 11, wherein the amplifying step for the target sequence in the 23S rRNA or DNA encoding the 23S rRNA further includes a third oligomer selected from the group consisting of SEQ ID Nos. 9 to 15 and 188, the completely complementary sequences of SEQ ID Nos. 9 to 15 and 188, and the RNA equivalent of SEQ ID Nos. 9 to 15 and 188, and wherein the third oligomer selected from this group includes a 3′ blocked end.

18. The method of claim 11, wherein the detecting step for the amplified product made from the target sequence in the 23S rRNA or DNA encoding the 23S rRNA uses a detection probe selected from the group consisting of SEQ ID Nos. 99 to 126 and 183, the completely complementary sequences of SEQ ID Nos. 99 to 126 and 183, and the RNA equivalent of SEQ ID Nos. 99 to 126 and 183.

19. The method of claim 11 further comprising a target capture step to purify the 23S rRNA or DNA encoding the 23S rRNA from the sample by hybridizing to the 23S rRNA or DNA encoding the 23S rRNA a target capture probe selected from the group consisting of SEQ ID Nos. 131 to 134.

20. The method of claim 11, wherein the amplified product is detected by using a detection probe that includes a label.
